SEQUENCE LISTING (15 sequences)

SEQ ID NO: 1 - DNA, 180 bp, Homo sapiens
ctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaa

SEQ ID NO: 2 - DNA, 220 bp, Homo sapiens
attcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaag

SEQ ID NO: 3 - DNA, 250 bp, Homo sapiens
ggtgacaattctccatttgatggacccaatggaatccttgctcatgcctttcagccaggccaaggtattggaggagatgctcattttgatgccgaagaaacatggaccaacacctccgcaaattacaacttgtttcttgttgctgctcatgaatttggccattctttggggctcgctcactcctctgaccctggtgccttgatgtatcccaactatgctttcagggaaaccagcaactactcactccctc

SEQ ID NO: 4 - DNA, 280 bp, Homo sapiens
atttttacttcactggtgtgcaagacatcaatgacaagaggttccaggagacctttgaggacgtattctctgaccgctcccttcgcaaagtgccctggtacgtgctagccggaaaccatgaccaccttggcaatgtctctgcccagattgcatactctaagatctccaagcgctggaacttccccagccctttctaccgcctgcacttcaagatcccacagaccaatgtgtctgtggccatttttatgctggacacagtgacactatgtggcaactcaga

SEQ ID NO: 5 - DNA, 220 bp, Homo sapiens
atgagaaagccctgaagagggcagtggcccgagtgggacctgtctctgtggccattgatgcaagcctgacctccttccagttttacagcaaaggtgtgtattatgatgaaagctgcaatagcgataatctgaaccatgcggttttggcagtgggatatggaatccagaagggaaacaagcactggataattaaaaacagctggggagaaaactggggaaa

SEQ ID NO: 6 - DNA, 230 bp, Homo sapiens
cggaaacagtgaatcaactcaaaaatgtggaatagatgttaccctgtgtgaggaggcattcttcaggtttgctgttcctacaaagtttacgcctaactggcttagtgtcttggtagacaatttgcctggcaccaaagtaaacgcagagagtgtagagaggataaaacggcaacacagctcacaagaacagactttccagctgctgaagttatggaaacatcaaaacaaag

SEQ ID NO: 7 - DNA, 240 bp, Homo sapiens
agaattaaacaggcctttcaaggagctgtgcaaaaggaattacaacatatcgttggatcacagcacatcagagcagagaaagcgatggtggatggctcatggttagatctggccaagaggagcaagcttgaagctcagccttttgctcatctcactattaatgccaccgacatcccatctggttcccataaagtgagtctgtcctcttggtaccatgatcggggttgggccaagatctcc

SEQ ID NO: 8 - DNA, 240 bp, Homo sapiens
ggcacaaactttcagagacagcagagcacacaagcttctaggacaagagccaggaagaaaccaccggaaggaaccatctcactgtgtgtaaacatgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatc

SEQ ID NO: 9 - DNA, 210 bp, Homo sapiens
atgggtattccagaagaaccaccttgtcttcaaaaatgtatcacaccaaaggacaagaaggttctgtttgtctccggtcatcagactgtgcctcaggattgtgttgtgctagacacttctggtccaagatctgtaaacctgtcctgaaagaaggtcaagtgtgtaccaagcataggagaaaaggctctcatggactagaaatattccagc

SEQ ID NO: 10 - DNA, 200 bp, Homo sapiens
gagagacaccagagaacccaccatggccccctttgagcccctggcttctggcatcctgttgttgctgtggctgatagcccccagcagggcctgcacctgtgtcccaccccacccacagacggccttctgcaattccgacctcgtcatcagggccaagttcgtggggacaccagaagtcaaccagaccaccttataccagc

SEQ ID NO: 11 - DNA, 230 bp, Homo sapiens
acttaaaggaggccggagctgtggggagctcagagctgagatcctacaggagtccagggctggagagaaaacctctgcgaggaaagggaaggagcaagccgtgaatttaagggacgctgtgaagcaatcatggatgcaatgaagagagggctctgctgtgtgctgctgctgtgtggagcagtcttcgtttcgcccagccaggaaatccatgcccgattcagaagaggagc

SEQ ID NO: 12 - DNA, 180 bp, Homo sapiens
gccccacccagaacctcttcctctccccatggagcatctcgtccaccatggccatggtctacatgggctccaggggcagcaccgaagaccagatggccaaggtgcttcagtttaatgaagtgggagccaatgcagttacccccatgactccagagaactttaccagctgtgggttcatgc

SEQ ID NO: 13 - DNA, 280 bp, Homo sapiens
agagccagcctggcgccccagcgggatcccagagaaggatctgcacagtgttatggcaccctacttcctgcagcaacgggacaccctgcggcgccatgtgcagaaacaggaggccgagaaccagcagctggcagatgccgtcctggcagggcggaggcaggtggaggagctgcagctacaggtccaggcccagcagcaggcctggcaggctctacacagagaacagagggagctggttgctgtgctgagggagcctgagtgaggagaccgccagccccag

SEQ ID NO: 14 - DNA, 420 bp, Homo sapiens
aatcttctctcagttgcttataaaaatgttgtaggagcccgtaggtcatcttggagggtcgtctcaagtattgaacaaaagacggaaggtgctgagaaaaaacagcagatggctcgagaatacagagagaaaattgagacggagctaagagatatctgcaatgatgtactgtctcttttggaaaagttcttgatccccaatgcttcacaagcagagagcaaagtcttctatttgaaaatgaaaggagattactaccgttacttggctgaggttgccgctggtgatgacaagaaagggattgtcgatcagtcacaacaagcataccaagaagcttttgaaatcagcaaaaaggaaatgcaaccaacacatcctatcagactgggtctggcccttaacttctctgtgttctattatgagatt

SEQ ID NO: 15 - DNA, 350 bp, Homo sapiens
ttggagggtcgtctcaagtattgaacaaaagacggaaggtgctgagaaaaaacagcagatggctcgagaatacagagagaaaattgagacggagctaagagatatctgcaatgatgtactgtctcttttggaaaagttcttgatccccaatgcttcacaagcagagagcaaagtcttctatttgaaaatgaaaggagattactaccgttacttggctgaggttgccgctggtgatgacaagaaagggattgtcgatcagtcacaacaagcataccaagaagcttttgaaatcagcaaaaaggaaatgcaaccaacacatcctatcagactgggtctggcccttaacttct

(19) United States
(12) Patent Application Publication
(10) Pub. No.: US 2016/0348176 A1
(43) Pub. Date: Dec. 1, 2016

(54) METHOD FOR DETERMINATION OF THE STATUS OF A DISEASE

(71) Applicant: NOBEL BIOCARE SERVICES AG, Kloten (CH)

(72) Inventor: Jan HALL, Goteborg (SE)

(21) Appl. No.: 15/116,588

(22) PCT Filed: Jan. 29, 2015
(86) PCT No.: PCT/EP2015/051783

§ 371 Date: Aug. 4, 2016

(30) Foreign Application Priority Data
Feb. 6, 2014 (GB) 1402036.6

(51) Int. Cl. C12Q 1/68; G01N 33/68

(52) U.S. Cl. CPC C12Q 1/6883; G01N 33/6893; C12Q 2600/158; C12Q 2600/112; G01N 2800/18; G01N 2800/245; G01N 2800/56

(57) ABSTRACT

The present invention relates to a method for determining the state of peri-implant disease comprising the steps of quantifying the expression level of one or more regulated markers of a group of markers forming a panel, said one or more regulated markers being related to the plasminogen system and/or inflammation and/or proteolytic activity or combinations thereof or ratios thereof in an ex vivo sample; and determining the state of peri-implant disease by comparing the expression level obtained in step a with a reference value. The present invention also relates to a kit for performing the invention.

### TECHNICAL FIELD

The present invention relates to a method for diagnosing the status of periodontal and peri-implant disease.

### BACKGROUND ART

A large number of dental implant rehabilitation procedures are performed every year. In contrast to the vast majority of cases where implant treatment is successful, a certain number of patients develop peri-implant disease. We distinguish between two states of peri-implant disease, mucositis, which involves soft tissue inflammation and peri-implantitis, which in addition to inflammation involves loss of the implant supporting bone. Mucositis and peri-implantitis may also be considered peri-implant tissue conditions. Both states may exhibit proteolytic activity, but at different magnitudes. In some cases, non-surgical treatment with mechanical debridement and flushing with 3% hydrogen peroxide may be a sufficiently effective treatment. In cases of persisting peri-implant disease, respective surgery in combination with surface debridement is often performed. By surgical correction of osseous defects (e.g. bone peaks) at the diseased implant site, pocket depths can be reduced and provide for a soft tissue morphology that facilitates oral hygiene. However, certain patients do not respond sufficiently well to treatment, and in spite of good plaque control and minimal inflammation of the peri-implant mucosa, symptoms including suppuration and progressive bone loss may recur in some cases. The reasons for such relapses are not known. A desire for improved understanding of the etiology of peri-implant disease and for the development of more sensitive diagnostic tools allowing for earlier detection and interventions is at hand; thus, increasing the predictability of implant treatment in susceptible patients. Moreover, in order to increase the survival rate of implants presenting signs of bone loss, clinical intervention at an early stage of disease progression is desirable. This requires early establishment of the status of the disease, and therefore, more sensitive techniques are required.

The diagnosis of peri-implant disease is generally based on clinical measurements combined with radiographic evidence of bone loss. Peri-implantitis is often clinically translated into formation and deepening of pockets, breakdown of the peri-implant epithelial seal, bleeding on probing (BoP), purulence and progressive bone loss.

These diagnostic methods are often used in combination for diagnosis of peri-implant disease as indicators of extensive pathologic changes in the implant-supporting tissue. The limited sensitivity and/or specificity of such diagnostic methods make early detection of pathologic changes difficult.

The viability of using analysis of genetic markers in the gingival crevicular fluid in plaque samples as a potential prognostic and diagnostic tool for peri-implant disease has been studied by a number of authors with variable results.

An often studied marker is Interleukin-1β (IL-1β), which is a pro-inflammatory cytokine involved in several biologic processes, including immune regulation, inflammation and connective tissue metabolism. IL-1β stimulates bone resorption and inhibits bone formation (Panagakos et al., Int J Oral Maxillofac Implants 1996, 11:794-799). IL-1β is produced mainly by macrophages but also by other cells including neutrophilic granulocytes. Several studies have shown the presence of IL-1β in the crevicular fluid around implants presenting signs of peri-implant disease. Significantly elevated levels have been reported for peri-implantitis compared to healthy sites (Panagakos et al., Int J Oral Maxillofac Implants 1996, 11:794-799; Kao et al., Int J Oral Maxillofac Implants 1995, 10:696-701; Murata et al., Clin Oral Impl Res 2002, 13:637-643) and compared to peri-implant mucositis sites (Murata et al., Clin Oral Impl Res 2002, 13:637-643), and also when comparing subjects with early and advanced signs of peri-implantitis. However, Hultin et al. (Clin Oral Impl Res 2002, 13:349-358) showed contradictory results with no difference in IL-1β expression between peri-implantitis and healthy sites.

Interleukin-8 (IL-8) is a pro-inflammatory marker and chemotactic factor for neutrophils. It participates in the regulation of the innate immune response to microbial invasion in periodontitis (Nassar et al., Infection and Immunity 2002, 268-276; Goutoudi et al., Int J Dent 2012; 2012:362905) and peri-implantitis (Petkovic et al., Int J Oral Maxillofac Surg 2010, 39(5):478-85). Nowzari et al. (Clin Implant Dent Relat Res 2008, 10(3):166-173) studied cytokine presence around implants and teeth in healthy subjects, and found a two-fold increase of IL-8 around implants compared with teeth.

Interleukin-6 (IL-6) is a multifunctional cytokine produced by various cells to regulate hematopoiesis, inflammation, immune responses, and bone homeostasis (Yoshitake et al., J Biol Chem 2008, 283:11535-11540). The level of IL-6 in saliva samples from subjects with peri-implant disease was significantly elevated compared with saliva samples from healthy subjects in a study by Liskmann et al. (Int J Oral Maxillofac Implants 2006, 21(4):543-50). Konttinen et al. (Int J Periodontics restorative Dent 2006, 26:135-141) measured statistically higher levels of IL-6 at failing implants with peri-implantitis compared with healthy implant sites.

Matrix metalloproteinases (MMPs) are proteolytic enzymes involved in degradation and removal of collagen from damaged tissue. MMPs are secreted by cells residing in the inflammatory sites in response to stimuli such as lipopolysaccharide and cytokines (Aboyoussef et al., Int J Oral Maxillofac Implants 1998, 13:689-696). Collagenases and gelatinases are two sub-families of the MMP superfamily. Findings by Kivelä-Rajamäki et al. (Clin Oral Impl Res 2003, 14:158-165) indicated that increased levels of MMP-8 (collagenase-2) may be associated with the active phase of inflammatory peri-implant disease. The expression of MMP-9 (gelatinase B) has also been studied; while Ma et al. (Clin Oral Impl Res 2003, 14:709-713) showed an association between MMP-9 and bone levels, Aboyoussef et al. (Int J Oral Maxillofac Implants 1998, 13:689-696) failed to show any significant differences between healthy and peri-implantitis sites.

The imbalance between MMPs and tissue inhibitors of matrix metalloproteinases (TIMPs) is considered to trigger the degradation of extracellular matrix, basement membrane, and alveolar bone, and thus to initiate periodontal disease (Sorsa et al., Oral Diseases 2004, 10: 311-318). It has been suggested that salivary MMP-8, TIMP-1 and especially their ratios are potential candidates in the detection of advanced periodontitis (Gursoy et al., Clin Periodontol 2010, 37:487-493).

The plasminogen system is of central importance in extracellular proteolysis in physiological as well as pathological tissue remodeling (reviewed by Collen, Thromb Haemost 1999, 82:259-270). Plasmin is a broadly active protease that is capable of degrading many extracellular proteins as well as activating latent collagenase and other metalloproteinase (Werb et al., New Eng J Med 1977, 296:1017-1023; Matrisian, Bioessays 1992, 14:455-463). Plasmin acts directly on the extracellular matrix (ECM) by cleaving non-collagenous ECM proteins and also indirectly by activating proforms of a whole range of other enzymes, among them the matrix metalloproteinases (MMPs), with specificity for different connective tissue proteins. Through the interaction between the plasminogen system and other tissue degrading systems, plasminogen represents an important dormant proteolytic potential, and strict control of its activation is important for maintaining the integrity of the tissues. Plasmin is formed from its inactive precursor plasminogen by plasminogen activators (serine proteases of which two types have been identified: urokinase type, u-PA, and tissue type, t-PA), which are specifically inhibited by the plasminogen activator inhibitors (PAI-1 and PAI-2), through the formation of bimolecular 1:1 covalent complexes. The levels of tPA as well as PAI-2 have been shown to be higher in gingival crevicular fluid (GCF) from inflamed than healthy sites (Kinnby, Biol Chem 2002, 383:85-92). A relatively increased level of PAI-2 has been associated with tissue-protective functions in pregnancy as well as periodontitis (Kinnby et al., J Periodont Res 1996, 31:271-277; Olofsson et al., J Periodont Res 2002, 37:60-65).

Different treatment alternatives for pen-implant disease have been proposed. It has been suggested that non-surgical therapy (e.g. surface debridement without access surgery) may be successful in cases of pen-implant mucositis, but appears to be less effective for sites presenting peri-implantitis (Renvert et al., J Clin Periodontol 2008, 35 (Suppl 8):305-315). Clinical data suggests that surgical treatment—e.g. open debridement including surface decontamination in combination with systemic antibiotics—may be a viable treatment option for peri-implantitis lesions (Claffey et al., J Clin Periodontol 2008, 35 (Suppl 8): 316-332). However, to date no common therapy exists, and advanced peri-implantitis remains difficult to treat.

The marginal bone around the implant crestal region is usually a significant indicator of implant health. The level of the crestal bone may be measured from the crestal position of the implant at the initial implant surgery. The most common method to asses bone loss is by radiographic evaluation. The bone level can thus be measured on the radiographs and can be defined as the distance from the junction between the fixture and its abutment to the crest of the marginal bone mesially and distally to the implants (Ahlqvist et al., Int J Oral Maxillofac Implants 1990, 5(2):155-163). Of course, conventional radiographics only monitor the mesial or distal aspect of bone loss around the implant body (Misch et al., Implant Dentistry 2008, 17(1):5-15). Lack of unambiguous information on ongoing bone loss may result in unnecessary or even incorrect treatment of peri-implant disease. The pen-implant bone level is determined from radiographs usually taken at the time of diagnosis. The bone level is compared with what is considered normal, and one or more radiographs taken at earlier time points are used to assess the bone loss. However, radiographs provide a stationary image of the bone situation; hence, evidence of bone demineralization does not necessarily imply ongoing disease activity. This holds true also for periodontal bone levels, and data on progression of periodontitis do not demonstrate a continuous process but instead bursts of activity (exacerbation), remission and periods of inactivity (Hall et al., Eur J oral Implantol 2011, 4(4):371-382). In addition, the limited sensitivity of radiographs seldom allows for detection of the very early stages of the pathological bone degradation processes involved in several diseases. Moreover, it is important that all radiographic examinations be performed using appropriate and reproducible projection techniques. The precision in measurements performed on radiographs is low, especially when related to small average bone loss, and it indicates the difficulties involved in the interpretation of them. Furthermore, the bone loss rate can only be measured within a long period of time, typically one year (Ahlqvist et al., Int J Oral Maxillofac Implants 1990, 5(2):155-163), and involves exposing patients to frequent radiation. Therefore, it seems likely that establishment of ongoing bone degradation in peri-implantitis and periodontitis patients is a prerequisite for increased precision of individualized patient treatment. See patent application GB1302257.9. The present invention disclosed here points to a different approach to establish the status of peri-implant disease.

### SUMMARY OF THE INVENTION

Since lack of unambiguous information on ongoing bone loss may result in unnecessary or even incorrect treatment of conditions that affect bone, it is highly desirable to quickly and precisely establish the peri-implant tissue condition for increased accuracy of individualized patient treatment and disease prognosis. The limited sensitivity of radiographs seldom allow for establishment of the treatment efficacy very early after the treatment has been provided to the patient. Obtained radiographs provide information on marginal bone levels at the time of examination, but they do not provide unambiguous establishment of ongoing bone degradation. Moreover, the limit of quantification for measurements of marginal bone level changes using conventional radiographs has previously been estimated to 0.47 mm (Ahlqvist et al., Int J Oral Maxillofac Implants 1990, 5(2):155-163). Therefore, it seems likely that a quick establishment of the peri-implant disease status in patients suffering from a condition that affects bone is a prerequisite for increased accuracy of patient diagnosis and treatment. This also avoids exposure of patients to frequent radiation.

The present invention thus provides a method for determining the state of peri-implant disease, wherein the method comprises the steps of:

a) quantifying the expression level of one or more regulated markers selected from the group consisting of tPA, IL-4, IL-6, IL-8, IL-1β, IL-10, IL-12, IL18, TIMP-1 and PAI-2, or any combination thereof, or any ratio thereof, and/or quantifying the expression levels of one or more of the following combinations of markers: tPA and tPA/PAI-2; PAI-2 and tPA/PAI-2; tPA, PAI-2 and tPA/PAI-2; IL-1β and IL-8; IL-8 and PAI-2; IL-6, TIMP-1 and PAI-2; tPA and PAI-2; IL-1β, IL-8 and IL-6; IL-1β, IL-8, IL-6 and TIMP-1; IL-1β, IL-8, IL-6, TIMP-1 and tPA; IL-1β, IL-8, IL-6, TIMP-1 and PAI-2; IL-1β, IL-8, PAI-2 and tPA; IL-1β, IL-8, and PAI-2; IL-1β, IL-8 and tPA; IL-1β, IL-8 and tPA/PAI-2; IL-1β, IL-8, TIMP-1 and PAI-2; IL-1β, IL-8, TIMP-1 and tPA; IL-1β and PAI-2; in an ex vivo sample; and
b) determining the state of peri-implant disease by comparing the expression level obtained in step a) with one or more reference expression level(s).

Moreover, the present invention provides a kit for carrying out the methods of the invention.

### TERMS AND ABBREVIATIONS

cDNA Complementary DNA
Cq Quantification cycle
DNA Deoxyribonucleic acid
ECM Extracellular matrix

### ELISA Enzyme Linked Immunosorbent Assay

GAPDH Glyceraldehyde-3-phosphate dehydrogenase
GCF Gingival crevicular fluid
HPRT1 Hypoxanthine-guanine phosphoribosyltransferase

### IL Interleukin

### MC Mucositis

MMPs Matrix metalloproteinases
mRNA Messenger RNA
PAI-2 Plasminogen activator inhibitor type 2 (SerpinB2)

### PI Peri-implantitis

PICF Peri-implant crevicular fluid
qPCR Quantitative Polymerase Chain Reaction
RNA Ribonucleic acid

### SEQ Sequence

TIMPs Tissue inhibitors of matrix metalloproteinases
tPA Tissue plasminogen activator

### UBC Ubiquitin C

uPA Urokinase plasminogen activator
YWHAZ Tyrosine 3/tryptophan 5-monoxygenase activation protein, zeta polypeptide

### BRIEF DESCRIPTION OF THE FIGURES

The expression levels referred to in figure captions 1-7 were measured in samples taken from subjects' peri-implant crevicular fluid (PICF).

FIG. 1: The expression levels of IL-1β value obtained are compared with the expression levels of PAI-2 (normalized expression in both cases) in a sample from a subject with peri-implantitis before and after treatment. The expression level of IL-1β is significantly lower and the expression level of PAI-2 remains similar after treatment, indicating that the treatment had an effect and the subject's peri-implant tissue is healthy.

FIG. 2: The expression levels of IL-8 value obtained is compared with the expression levels of PAI-2 in a sample from a subject with peri-implantitis before and after treatment. The expression levels of IL-8 and PAI-2 have not changed after treatment and the peri-implantitis condition is unaltered.

FIG. 3: The expression levels of IL-6, TIMP-1 and PAI-2 are oppositely regulated in subjects with mucositis compared with peri-implantitis (normalized expression).

FIG. 4: The expression level of tPA is oppositely regulated in subjects with mucositis compared with peri-implantitis (normalized expression).

FIG. 5: Probability for peri-implantitis compared to mucositis as a function of normalized Cq-values for tPA.

FIG. 6: Probability for peri-implantitis compared to healthy peri-implant conditions as a function of normalized Cq-values for IL-1β.

FIG. 7: Probability for mucositis compared to healthy peri-implant conditions as a function of normalized Cq-values for tPA.

FIG. 8: The expression levels of IL-8 value obtained is compared with the expression levels of tPA in a sample from a subject with peri-implantitis before and after treatment. The expression levels of IL-8 and tPA have not changed after treatment and the peri-implantitis condition is unaltered.

### DETAILED DESCRIPTION OF THE INVENTION

The detailed description discloses specific and/or preferred variants of the individual features of the invention. The present invention also contemplates as particularly preferred embodiments those embodiments, which are generated by combining two or more of the specific and/or preferred variants described for two or more of the features of the present invention. The present invention provides a method for determining the state of peri-implant disease. The method comprises the steps of a) quantifying the expression level of one or more (regulated) markers of a group of markers forming a panel, said one or more regulated markers being related to the plasminogen system, inflammation and/or proteolytic activity or ratio thereof in an ex vivo sample; and b) determining the state of peri-implant disease by comparing the expression level obtained in step a) with a reference value (reference expression level(s)).

Biomarkers (markers hereafter) may be defined as substances that are measured objectively and evaluated as an indicator of normal biologic processes, pathogenic processes and pharmacologic responses to a therapeutic intervention. Biomarkers are molecules that may be used to monitor health status, disease onset, treatment response and outcome (Zia et al., Biology and medicine 2011, 3(2):45-52).

The marker or combination thereof, or ratio thereof related to the plasminogen system, inflammation and proteolytic activity is not particularly limited and may be one or more of IL-1β, and/or IL-10, and/or IL-8, and/or IL-6, and/or MMP-8, and/or MMP-2, and/or MMP-9 and/or TIMP-1, and/or tPA, and/or PAI-2, or combinations thereof. Preferred are IL-1β, IL-8, TIMP-1 and PAI-2. Preferred are also IL-1β, IL-4, IL-8, tPA and PAI-2. Preferred are also tPA and PAI-2. Preferred is also the ratio tPA/PAI-2. tPA is preferred. The preferred markers of the present invention may be identified by the following accession numbers (National Center for Biotechnology Information, NCBI):

### Interleukin-1β (IL-1β): NM_000576.2

### Interleukin-8 (IL-8): NM_000584.3

### Interleukin-10 (IL-10): NM_000572.2

### Interleukin-4 (IL-4): M23442.

Tissue inhibitor of matrix metalloproteinase (TIMP-1): NM_003254.2
Plasminogen activator inhibitor type 2 (serpinB2) (PAI-2): NM_001143818.1
Tissue plasminogen activator (tPA): AY221101

For example, said one or more (regulated) markers are selected from the group consisting of tPA, IL-1β, IL-4, IL-6, IL-8, IL-10, IL-12, IL18, MMP-2, MMP-8, MMP-9, TIMP-1 and PAI-2, or any combination thereof, or any ratio thereof. Preferably, said one or more (regulated) markers are selected from the group consisting of tPA, IL-1β, PAI-2 and IL-8. Preferably, said regulated marker is tPA.

Preferably, the combinations of said markers are
the following:

| | |
|---|---|
| tPA and tPA/PAI-2 | PAI-2 and tPA/PAI-2 |
| tPA, PAI-2 and tPA/PAI-2 | IL-1β and IL-8 |
| IL-8 and PAI-2 | IL-8 and tPA |
| IL-6, TIMP-1 and PAI-2 | tPA and PAI-2 |
| tPA and IL-1β | tPA and TIMP-1 |
| IL-1β, IL-8 and IL-6 | IL-1β, IL-8, IL-6 and TIMP-1 |
| IL-1β, IL-8, IL-6, TIMP-1 and tPA | IL-1β, IL-8, IL-6, TIMP-1 and PAI-2 |
| IL-1β, IL-8, PAI-2 and tPA | IL-1β, IL-8, and PAI-2 |
| IL-1β, IL-8 and tPA | IL-1β, IL-8 and tPA/PAI-2 |
| IL-1β, IL-8, TIMP-1 and PAI-2 | IL-1β, IL-8, TIMP-1 and tPA |
| IL-1β and PAI-2 | |

The ratio of said markers can be for example tPA/PAI-2 and/or IL-1β/PAI-2.

Preferably, the ratio of said markers is the following:
tPA/PAI-2

The information provided by the above ratios may also be obtained from the calculation of the inverse ratios (e.g. PAI-2/tPA).

For example, the method of the invention is a method for determining the state of peri-implant disease, wherein the method comprises the steps of:

a) quantifying the expression level of one or more regulated markers selected from the group consisting of tPA, IL-1β, IL-4, IL-6, IL-8, IL-10, IL-12, IL18, TIMP-1 and PAI-2, or any combination thereof, or any ratio thereof in an ex vivo sample; and
b) determining the state of peri-implant disease by comparing the expression level obtained in step a) with one or more reference expression level(s).

For example, said one or more regulated markers is tPA. For example, said one or more regulated markers or ratio thereof is tPA/PAI-2.

For example, said one or more regulated markers are selected from the group consisting of IL-1β and IL-8, or any combination thereof, or any ratio thereof. For example, said one or more regulated markers are IL-1β and IL-8.

For example, said one or more regulated markers are selected from the group consisting of IL-8 and PAI-2, and/or IL-8 and tPA or any combination thereof, or any ratio thereof. For example, said one or more regulated markers consist of IL-8 and PAI-2. For example, said one or more regulated markers consist of IL-8 and tPA.

Preferably, the method of the invention is a method for determining the state of peri-implant disease, wherein the method comprises the steps of:

a) quantifying the expression level of one or more regulated markers selected from the group consisting of tPA, IL-4, IL-6, IL-10, IL-12, IL18, TIMP-1 and PAI-2, or any combination thereof, or any ratio thereof in an ex vivo sample; and
b) determining the state of peri-implant disease by comparing the expression level obtained in step a) with one or more reference expression level(s).

For example, said one or more regulated markers are selected from the group consisting of IL-6, TIMP-1 and PAI-2, or any combination thereof, or any ratio thereof. For example, said one or more regulated markers consist of IL-6, TIMP-1 and PAI-2.

Preferably, said one or more regulated markers are selected from the group consisting of tPA and PAI-2, or any combination thereof, or any ratio thereof.

Preferably, said one or more regulated markers consist of tPA and PAI-2.

Preferably, the expression level of the one or more regulated markers is the ratio between tPA and PAI-2 (tPA/PAI-2).

Preferably, the expression level of the one or more regulated markers is tPA, namely, the method comprises quantifying the expression level of tPA in step (a).

Optionally, the method of the present invention further comprises saving the information regarding expression level of one or more regulated markers obtained in step (a) and/or saving the information regarding the state of peri-implant disease obtained in step (b) in a data carrier, such as for example in an electronic data carrier.

The inventors have shown that the levels of one or more markers related to the plasminogen system, inflammation and proteolytic activity or a ratio between two or more of them are related to the state of pen-implant disease. This allows for a quick and sensitive determination of mucositis and/or peri-implantitis, which was not possible to perform before this invention. The markers can be indicative of the presence or absence of a condition that affects bone. Furthermore, the state of peri-implant disease can indicate that the patient should undergo a certain treatment.

Mucositis and/or peri-implantitis can also be referred to as peri-implant tissue conditions.

The present invention provides a method for a quick establishment of the state of peri-implant disease, preventing the patient from undergoing unnecessary radiation exposure and providing the clinician with valuable information in order to diagnose, select the suitable therapy and/or estimate the prognosis of the implant.

For example, the present invention provides a method for the diagnosis of peri-implant disease, such as mucositis or peri-implantitis. For example, the present invention provides a method for the diagnosis of the peri-implant tissue condition, such as mucositis or peri-implantitis.

For example, the present invention provides a method for determining the prognosis of peri-implant disease, such as mucositis or peri-implantitis. For example, the present invention provides a method for determining the prognosis of the peri-implant tissue condition, such as mucositis or peri-implantitis.

In the context of the present invention, the state of peri-implant disease may be defined as mucositis or peri-implantitis without using a measurement of the ongoing bone degradation. In other words, the state of the disease may be defined as the difference of the expression levels of one or more markers related to the plasminogen system and/or the expression levels of one or more inflammatory and/or proteolytic activity markers. In the particular case of dental implants, the status of the disease may be defined as mucositis if one or more of IL-1β, IL-8, IL-6 and TIMP-1 markers are down-regulated and/or if PAI-2 is up-regulated compared with the same markers in the peri-implantitis case. In the particular case of dental implants, the condition of the peri-implant tissue may be defined as mucositis if one or more of IL-1β, IL-8, IL-6 and TIMP-1 markers are down-regulated and/or if PAI-2 is up-regulated compared with the same markers in the peri-implantitis case.

In the particular case of dental implants, the status of the disease may be defined as mucositis if one or more of IL-1β, IL-8, IL-6 and TIMP-1 markers are down-regulated and/or if tPA is up-regulated compared with the same markers in the peri-implantitis case. In the particular case of dental implants, the condition of the peri-implant tissue may be defined as mucositis if one or more of IL-1β, IL-8, IL-6 and TIMP-1 markers are down-regulated and/or if tPA is up-regulated compared with the same markers in the peri-implantitis case

In the particular case of dental implants, the status of the disease may be defined as peri-implantitis if the marker IL-6 is up-regulated compared with the same marker in the mucositis and/or healthy cases. In the particular case of dental implants, the condition of the peri-implant tissue may be defined as peri-implantitis if the marker IL-6 is up-regulated compared with the same marker in the mucositis and/or healthy cases.

In the particular case of dental implants, the status of the disease may be defined as mucositis if tPA is up-regulated compared with the same marker in the peri-implantitis case. In addition, in the particular case of dental implants, the condition of the peri-implant tissue may be defined as mucositis if tPA is up-regulated compared with the same markers in the peri-implantitis case.

In addition, in the particular case of dental implants, the status of the disease may be defined as peri-implantitis if one or more of IL-1β, IL-8, IL-6 and TIMP-1 markers are up-regulated and/or if PAI-2 is down-regulated compared with the same markers in the mucositis case. In addition, in the particular case of dental implants, the condition of the peri-implant tissue may be defined as peri-implantitis if one or more of IL-1β, IL-8, IL-6 and TIMP-1 markers are up-regulated and/or if PAI-2 is down-regulated compared with the same markers in the mucositis case.

In addition, in the particular case of dental implants, the status of the disease may be defined as peri-implantitis if one or more of IL-1β, IL-8, IL-6 and TIMP-1 markers are up-regulated and/or if tPA is down-regulated compared with the same markers in the mucositis case. In addition, in the particular case of dental implants, the condition of the peri-implant tissue may be defined as peri-implantitis if one or more of IL-1β, IL-8, IL-6 and TIMP-1 markers are up-regulated and/or if tPA is down-regulated compared with the same markers in the mucositis case.

In addition, in the particular case of dental implants, the status of the disease may be defined as peri-implantitis if IL-1β and IL-8 markers are up-regulated and/or if tPA and PAI-2 are down-regulated compared with the same markers in the mucositis case. In addition, in the particular case of dental implants, the condition of the peri-implant tissue may be defined as peri-implantitis if IL-1β and IL-8 markers are up-regulated and/or if tPA and PAI-2 are down-regulated compared with the same markers in the mucositis case.

In the particular case of dental implants, the status of the disease may be defined as peri-implantitis if tPA is down-regulated compared with the same marker in the mucositis case. In the particular case of dental implants, the condition of the peri-implant tissue may be defined as peri-implantitis if tPA is down-regulated compared with the same markers in the mucositis case.

In the particular case of dental implants, the condition of peri-implantitis may be diagnosed if one or more of IL-1β and IL-8 markers are up-regulated compared with the same markers in the healthy tissue. For example, the condition of peri-implantitis may be diagnosed if IL-1β and IL-8 markers are up-regulated compared with the same markers in the healthy tissue.

In the particular case of dental implants, the condition of the pen-implant tissue may be defined as peri-implantitis if one or more of IL-1β and IL-8 markers are up-regulated compared with the same markers in the healthy tissue. For example, the condition of the peri-implant tissue may be defined as peri-implantitis if IL-1β and IL-8 markers are up-regulated compared with the same markers in the healthy tissue.

In the particular case of dental implants, the condition of mucositis may be diagnosed if the marker tPA is up-regulated compared with the same marker in the healthy tissue. In the particular case of dental implants, the condition of the peri-implant tissue may be defined as mucositis if the marker tPA is up-regulated compared with the same marker in the healthy tissue.

In the particular case of dental implants, the condition of the peri-implant tissue may be defined as mucositis if the ratio of markers tPA/PAI-2 is down-regulated compared with the same ratio of markers in the healthy tissue and/or in the peri-implantitis case. In the particular case of dental implants, the condition of mucositis may be diagnosed if the ratio of markers tPA/PAI-2 is down-regulated compared with the same ratio of markers in the healthy tissue and/or in the peri-implantitis case.

Markers for the plasminogen system, such as tPA, and/or PAI-2, and/or combinations thereof, and/or ratios thereof may be used to distinguish between mucositis and peri-implantitis, and/or to diagnose peri-implant tissue conditions such as peri-implantitis and/or mucositis.

Markers for inflammation, such as IL-4, and/or IL-1β, and/or IL-8, and/or IL-10, and/or IL-12, and/or IL18 and/or combinations thereof, and/or ratios thereof may be used to distinguish between mucositis and peri-implantitis, and/or to diagnose peri-implant tissue conditions such as peri-implantitis and/or mucositis.

Markers for proteolytic activity inhibitors, such as TIMP-1 may be used to distinguish between mucositis and peri-implantitis, and/or to diagnose peri-implant tissue conditions such as peri-implantitis and/or mucositis.

The ex vivo sample is preferably a body fluid or a tissue. The body fluid can be an oral fluid, and/or serum, and/or plasma, and/or cerebrospinal fluid, and/or synovial fluid, and/or peritoneal fluid, and/or blood, and/or saliva, preferably gingival crevicular fluid and more preferably peri-implant crevicular fluid.

The tissue can be bone, and/or a tissue adjacent to the bone, and/or connective tissue, and/or medulla, and/or cartilage, and/or gingiva, and/or mucosa, and/or implant-supporting tissue, and/or bone adjacent to an implant, and/or bone adjacent to a tooth.

Preferably, the ex vivo sample is a body fluid, preferably an oral fluid, preferably gingival crevicular fluid and more preferably peri-implant crevicular fluid.

The ex vivo sample in which the markers for inflammation and/or proteolytic activity are measured may be obtained from the body fluid or tissue of the subject. Preferably, the ex vivo sample is obtained by inserting one or more sterile absorbents such as sterile paper points to the base of the pen-implant sulcus/pocket an left in situ for at least 60 seconds. Preferably, three or more sterile paper points are inserted in the sulcus/pocket.

Preferably, the expression level of two, three, four, five, six or more markers or ratio thereof is measured in order to obtain more accurate information on the status of peri-implant disease.

For example, the expression level of tPA is measured in order to obtain information on the status of peri-implant disease. For example, the expression level of PAI-2 is measured in order to obtain information on the status of peri-implant disease. For example, the expression level of tPA/PAI-2 is measured in order to obtain information on the status of peri-implant disease. For example, the expression levels of IL-1β and IL-8 are measured in order to obtain more accurate information on the status of peri-implant disease. For example, the expression levels of IL-1β, IL-8 and PAI-2 are measured in order to obtain information on the status of peri-implant disease. For example, the expression levels of IL-1β, IL-8 and tPA are measured in order to obtain information on the status of peri-implant disease. For example, the expression levels of IL-1β, IL-8 and tPA/PAI-2 are measured in order to obtain information on the status of peri-implant disease. For example, the expression levels of IL-1β, IL-8, tPA and PAI-2 are measured in order to obtain information on the status of peri-implant disease. For example, the expression levels of tPA and tPA/PAI-2, and/or PAI-2 and tPA/PAI-2 and/or tPA, PAI-2 and tPA/PAI-2 are measured in order to obtain information on the status of peri-implant disease. For example, the expression levels of tPA and IL-8, and/or PAI-2 and IL-8 are measured in order to obtain information on the status of peri-implant disease. For example, the expression levels of IL-1β and PAI-2 are measured in order to obtain information on the status of peri-implant disease.

For example, the expression level of tPA is measured in order to obtain information on the peri-implant condition. For example, the expression level of PAI-2 is measured in order to obtain information on the peri-implant condition. For example, the expression level of tPA/PAI-2 is measured in order to obtain information on the pen-implant condition. For example, the expression levels of IL-1β and IL-8 are measured in order to obtain more accurate information on the pen-implant condition. For example, the expression levels of IL-1β, IL-8 and PAI-2 are measured in order to obtain information on the pen-implant condition. For example, the expression levels of IL-1β, IL-8 and tPA are measured in order to obtain information on the peri-implant condition. For example, the expression levels of IL-1β, IL-8 and tPA/PAI-2 are measured in order to obtain information on the peri-implant condition. For example, the expression levels of IL-1β, IL-8, tPA and PAI-2 are measured in order to obtain information on the peri-implant condition. For example, the expression levels of tPA, and/or tPA and tPA/PAI-2, and/or PAI-2 and tPA/PAI-2 and/or tPA and/or PAI-2 and tPA/PAI-2 and/or IL-8 and tPA are measured in order to obtain information on the status of peri-implant condition. For example, the expression levels of tPA and IL-8, and/or PAI-2 and IL-8 are measured in order to obtain information on the status of peri-implant condition.

The method for quantifying the expression levels of one or more markers or ratio thereof related to inflammation and/or proteolytic activity in the ex vivo sample is not particularly limited and may be selected from a method of quantifying nucleic acids such as mRNA and/or a method for quantifying proteins such as RT-qPCR, hereafter referred to as qPCR, and/or Northern Blot, and/or immunoassay, and/or ELISA, and/or radioimmunoassay, and/or magnetic immunoassay, and/or fluorescent immunoassay, and/or immunoprecipitation, and/or surface plasmon resonance, and/or Western Blot, and/or immunohistochemistry or any combination thereof. A preferred method for quantification is qPCR. Experimental procedures typically include sample-processing steps (i.e. extraction).

The quantification of the expression levels of one or more markers or ratio thereof may be normalized by one or more reference genes. Normalization involves reporting the ratios of the expression level of the genes of interest to those of the reference genes. The reference genes can be selected using the freely available NormFinder program. Preferably, the reference genes are those which are stably expressed and their abundances show a strong correlation with the total amount of sample (in the case of qPCR, with the total amount of mRNA). More preferably, the reference genes are selected from GAPDH, YWHAZ, UBC and/or HPRT-1, among which YWHAZ and UBC are preferred.

The preferred reference genes of the present invention may be identified by the following accession numbers:

YWHAZ (Reference gene): NM_001135702.1
UBC (Reference gene): NM_001135702.1

In the context of the present application, expression level of a marker may mean (i) concentration, or (ii) detection signal specific for a marker, or (iii) a value that relates to (i) and/or (ii) by mathematical transformation.

In the method of the present invention the state of peri-implant disease (or the condition of the peri-implant tissue) may be determined by comparing the expression level obtained in step (a) with one or more reference expression level(s) (also referred to as reference value).

The one or more reference expression level(s) may be the expression level(s) of said one or more regulated markers, or any combination thereof, or any ratio thereof from an ex vivo sample taken from the same source at a different point in time.

The one or more reference expression level(s) may be the expression level(s) of said one or more regulated markers, or any combination thereof, or any ratio thereof from an ex vivo sample taken from a source showing a history of peri-implantitis.

The one or more reference expression level(s) may be the expression level(s) of said one or more regulated markers, or any combination thereof, or any ratio thereof from an ex vivo sample taken from a source showing a history of mucositis.

The one or more reference expression level(s) may be the expression level(s) of said one or more regulated markers, or any combination thereof, or any ratio thereof from an ex vivo sample taken from a source showing a history of healthy tissue.

In the case of qPCR, relative gene expression levels are preferably calculated using the ΔΔCq method (Livak et al., Methods 2001, 25:402-408) for each assay and by normalizing gene expression of each gene by the reference genes. The reference genes may be for example selected using the freely available NormFinder program (www.mdl.dk/publicationsnormfinder.htm. October 2010). The normalized gene expression can then be calculated for each subject using the following expression after logarithmic transformation: normalized expression of gene g=(Cq(n)−Cq(g)), where Cq(g) is the number of amplification cycles for gene g, and Cq(n) is the normalization factor (mean number of amplification cycles for the selected reference gene or genes) for the sample taken from the subject.

In the case of qPCR, the expression level of one or more markers or ratio thereof related to the plasminogen system, inflammation and/or proteolytic activity may be also quantified by quantification of the corresponding amplicon. An amplicon may be defined a piece of DNA or RNA that is the source and/or product of natural or artificial amplification or replication events. In the case of the present invention, the preferred amplicons for the quantification of the expression level of the one or more markers or ratio thereof related to inflammation and proteolytic activity or reference genes are the following:

### Interleukin-1β (IL-1β): SEQ ID NO 1

### Interleukin-6 (IL-6): SEQ ID NO 2

Matrix metalloproteinase-8 (MMP8) SEQ ID NO 3

### Interleukin-8 (IL-8): SEQ ID NO 8

Tissue inhibitor of matrix metalloproteinase (TIMP-1): SEQ ID NO 10
Tissue plasminogen activator (tPA): SEQ ID NO 11
Plasminogen activator inhibitor type 2 (serpinB2) (PAI-2): SEQ ID NO 12
YWHAZ (Reference gene): SEQ ID NO 14
UBC (Reference gene): SEQ ID NO 15

The methods of the present invention may be used for indicating the presence or absence of a condition that affects bone, preferably of a condition that affects bone surrounding implants and/or teeth. More preferably, said condition is peri-implant disease, and/or periodontal disease, among which peri-implant disease is preferred.

Peri-implant disease (also called peri-implantitis in presence of bone degradation) is defined as an inflammatory process affecting the tissue around an implant in function that has resulted in loss of supporting bone (Becker et al., Int J Oral Maxillofac Implants 1990, 5:31-38). Mucositis is often referred to as soft tissue inflammation, swelling, bleeding on probing and in some cases, suppuration, but with no signs of bone loss.

The methods of the present invention may be also used for evaluation of the prognosis of an implant or tooth.

The present inventors investigated the association between the expression level of certain markers or ratio between two or more markers or combination thereof that are related to the plasminogen system, inflammation and proteolytic activity, enabling the use as diagnostic factor for conditions that affect bone. Thus, by the method of the invention, an individual patient can be diagnosed to suffer or not to suffer from a condition that affects bone, preferably a condition that affects bone surrounding implants and/or teeth.

For example, by the methods of the invention, an individual patient can be diagnosed to suffer or not to suffer from mucositis and/or peri-implantitis. The inventors have shown that the expression level of certain markers or ratio between two or more markers that are related to the plasminogen system, inflammation and proteolytic activity in peri-implant crevicular fluid obtained from patients that have undergone implant treatments is related to the state of peri-implant disease. This method provides a quick diagnosis of the state of the peri-implant disease and to select the appropriate treatment.

The inventors have shown that the expression level of certain markers or ratio between two or more markers that are related to the plasminogen system, inflammation and proteolytic activity in peri-implant crevicular fluid obtained from patients that have undergone implant treatments is related to the condition of the peri-implant tissue. This method provides a quick diagnosis of the condition of the peri-implant tissue and to select the appropriate treatment.

It is thus not necessary to expose the subjects to radiation, and the clinician does not have to wait until the bone degradation has progressed to a measurable level assessed by radiographs to select a treatment and establish a prognosis of the patient.

The ex vivo sample in which the status of the disease and/or the condition of the peri-implant tissue is measured may be obtained from a patient which may or may not suffer from a condition that affects bone, preferably from a condition that affects bone surrounding implants and/or teeth. The preferred patient is a patient with one or more dental implants.

Preferably, the patient suffers and/or is likely to suffer from a condition that affects implant supporting tissue, and/or a condition that affects bone supporting implants, and/or a condition that affects the tissues around teeth, such as peri-implantitis, and/or mucositis, and/or periodontitis, and/or gingivitis or a combination thereof. More preferably, said patient suffers and/or is likely to suffer from peri-implant disease. For example, said patient suffers and/or is likely to suffer from mucositis. For example, said patient suffers and/or is likely to suffer from peri-implantitis.

The expression levels of markers for the plasminogen system and/or inflammation and/or proteolytic activity, for example, IL-1β, IL-8, TIMP-1, PAI-2 and/or tPA in an ex vivo sample of a patient may be quantified by qPCR.

The determination of how one or more of the three former markers (IL-1β, IL-8, TIMP-1) are regulated relative to PAI-2 will establish if the patient has mucositis or peri-implantitis. The determination of how one or more of the three former markers (IL-1β, IL-8, TIMP-1) are regulated relative to tPA will establish if the patient has mucositis or peri-implantitis. The patient can be his/her own control (reference expression level(s) (reference value)). In another example, the same markers are compared with the expression levels of the same markers taken from a healthy implant site, and/or a site with mucositis and/or a site with peri-implantitis from another patient or other patients. Preferably, this information is indicative of an appropriate treatment and/or disease prognosis.

Up-regulation of PAI-2 and similar expression level(s) of IL-1β and/or IL-8 and/or TIMP-1 compared with the expression levels of the same marker(s) from healthy PICF and/or peri-implant tissue may be indicative of a treatment comprising standard of care oral hygiene treatment and a maintenance program.

Up-regulation of tPA and similar expression level(s) of IL-1β and/or TIMP-1, and/or IL-8 compared with the expression levels of the same marker(s) from healthy PICF and/or peri-implant tissue may be indicative of a treatment comprising standard of care oral hygiene treatment and a maintenance program.

Up-regulation of IL-1β and/or IL-8 and/or TIMP-1 and similar expression levels of PAI-2 compared with the expression levels of the same marker(s) from healthy PICF and/or peri-implant tissue may be indicative of a treatment comprising standard of care oral hygiene treatment and a maintenance program and a follow up visit (with the clinician/dentist) within a few weeks.

Up-regulation of IL-1β and/or TIMP-1, and/or IL-8 and similar expression levels of tPA compared with the expression levels of the same marker(s) from healthy PICF and/or peri-implant tissue may be indicative of a treatment comprising standard of care oral hygiene treatment and a maintenance program and a follow up visit (with the clinician/dentist) within a few weeks.

If the up-regulation of IL-1β and/or IL-8 and/or TIMP-1 persists after the first or more follow up visits it may be indicative of a surgical treatment.

If similar levels of PAI-2 and/or tPA and up-regulation of IL-1β and/or IL-8 and/or TIMP-1 compared with the expression levels of the same marker(s) from healthy PICF and/or peri-implant tissue persist after the first or more follow up visits (with the clinician/dentist) it may be indicative of a surgical treatment.

If the expression level of PAI-2 is not different from IL-1β and/or IL-8 and/or TIMP-1 compared with a reference from healthy pen-implant tissue, it might be indicative of a standard program for oral hygiene.

If the expression level of tPA and/or IL-1β and/or TIMP-1, and/or IL-8 is similar to the expression levels of the same marker(s) from healthy PICF and/or peri-implant tissue, it might be indicative of a standard program for oral hygiene.

If the expression level of tPA/PAI-2 is down regulated compared with the expression levels of the same marker(s) from healthy PICF and/or peri-implant tissue, it might be indicative of mucositis and thus, of a standard program for oral hygiene. Persisting down regulation of tPA/PAI-2 and up regulation of IL-1β and/or IL-8 might be indicative of peri-implantitis and need for surgical treatment.

If the peri-implant tissue shows signs of redness and inflammation, the clinician (dentist) may have a first indication of mucositis. However, these indications are not enough proof of the condition of the patient (mucositis). With the method of the present invention, if the expression levels of tPA and/or PAI-2 are up-regulated and/or the expression levels of tPA/PAI-2 is down regulated compared with the expression levels of the same marker(s) from healthy PICF and/or peri-implant tissue, the clinician (dentist) is provided with information, indicative of mucositis and thus, recommends the patient to follow a standard program for oral hygiene.

If the peri-implant tissue shows signs of redness, inflammation and suppuration, the clinician (dentist) may have a first indication of peri-implantitis. However, these indications are not enough proof of the condition of the patient (peri-implantitis). With the method of the invention, if the expression levels of tPA and/or PAI-2 and/or tPA/PAI-2 are similar (i.e. their expression levels do not vary) compared with the expression levels of the same marker(s) from healthy PICF and/or peri-implant tissue, the clinician (dentist) is provided with information indicative of peri-implantitis and thus, recommends the patient to follow a treatment comprising standard of care oral hygiene treatment and a maintenance program, and a follow up visit within a few weeks.

If the redness, swelling and suppuration persist, the clinician (dentist) may have a first indication of peri-implantitis. However, these indications may not be enough proof of the condition of the patient (peri-implantitis). With the method of the invention, if the expression levels of tPA and/or PAI-2 and/or tPA/PAI-2 are similar (i.e. their expression levels do not vary) compared with the expression levels of the same marker(s) from healthy PICF and/or peri-implant tissue, the clinician (dentist) is provided with information indicative of peri-implantitis and need for surgical treatment.

The terms “human subject”, “subject” and “patient” are used interchangeably in the application. The terms “condition” and “disease” are used interchangeably in the application.

Further, the present invention provides a kit for carrying out the methods of the invention. The expression levels of inflammatory markers and/or markers for the plasminogen system, proteolytic activity indicative for a certain treatment (e.g. tPA, IL-1β, IL-6, IL-8, IL-4, IL-10, IL-12, IL18, TIMP-1 and PAI-2, or any combination thereof, or any ratio thereof) may be provided with the kit. With the help of the kit, the values of markers for the plasminogen system and/or inflammation and/or proteolytic activity in a sample taken from a patient can be compared. The kit of the invention might comprise a sample collection device, which is not limited and is a device for taking samples such as body fluids or tissue. Preferably, the sample collecting device is used for taking samples of pen-implant crevicular fluid. The sample collection device may be absorbents such as sterile paper points and/or a syringe and/or a biopsy device. Preferably, the sample collection devices are sterile absorbents, more preferably sterile paper points.

The kit of the present invention may further comprise a preservation medium for preserving the sample. The purpose of the preservation medium is to preserve biological samples, and may be any medium formulated to maintain the integrity and viability of the samples for downstream analysis. Preferably, the preservation medium may comprise inhibitors of RNases. Most preferably, the preservation medium is RNALater preservation medium (Qiagen, Hilden, Germany).

The kit of the present invention may also contain instructions on how to perform the method of the invention.

The kit of the present invention may further contain a box to send the sample to a central laboratory, where the expression levels of one or more markers related to the activity of the plasminogen system and/or inflammation and/or proteolytic activity or combination thereof are compared with the expression of the same markers in samples taken from healthy implant sites and/or sites with mucositis and/or sites with peri-implantitis. The comparison of the expression level values may be performed in the central laboratory.

Alternatively, the kit of the present invention may contain the necessary elements to quantify the expression levels of one or more markers related to the plasminogen system, inflammation and/or proteolytic activity or combinations thereof, or ratios thereof, such as stated above (for example tPA, IL-1β, IL-4, IL-6, IL-8, IL-10, IL-12, IL18, MMP-2, MMP-8, MMP-9, TIMP-1 and/or PAI-2, or any combination thereof, or any ratio thereof). In this case, the kit may comprise at least one detectable label and at least one substrate which specifically recognize one or more markers related to the plasminogen system, inflammation and/or proteolytic activity or combination thereof. For example, the kit may contain the necessary elements to compare the expression level of PAI-2 with one or more of IL-1β, IL-8 and/or TIMP-1 at sampling site.

Preferably, the kit contains the necessary
elements to quantify the expression levels of the IL-1β
following markers and/or combinations thereof
and/or ratio thereof:

| | |
|---|---|
| tPA | IL-6 |
| PAI-2 | IL-8 |
| IL-4 | IL-12 |
| IL-10 | MMP-2 |
| IL18 | MMP-9 |
| MMP-8 | IL-1β, IL-8 and IL-6 |
| TIMP-1 | IL-1β, IL-8, IL-6 and TIMP-1 |
| tPA and tPA/PAI-2 | IL-1β, IL-8, IL-6, TIMP-1 and tPA |
| PAI-2 and tPA/PAI-2 | IL-1β, IL-8, IL-6, TIMP-1 and PAI-2 |
| tPA, PAI-2 and tPA/PAI-2 | IL-1β, IL-8, PAI-2 and tPA |
| IL-1β and IL-8 | IL-1β, IL-8, and PAI-2 |
| IL-8 and PAI-2 | IL-1β, IL-8 and tPA |
| IL-8 and tPA | IL-1β, IL-8 and tPA/PAI-2 |
| IL-6, TIMP-1 and PAI-2 | IL-1β, IL-8, TIMP-1 and PAI-2 |
| tPA and PAI-2 | IL-1β, IL-8, TIMP-1 and tPA |
| tPA and IL-1β | tPA/PAI-2 |
| tPA and TIMP-1 | |
| IL-1β/PAI-2 | |

For example, the kit may contain the necessary elements to compare the expression level of tPA with one or more of IL-1β, IL-8 and/or TIMP-1 at sampling site.

For example, the kit may contain the necessary elements to compare the expression level of tPA and/or PAI-2 with the expression levels of tPA/PAI-2 at sampling site.

For example, the kit may contain the necessary elements to quantify the expression levels of the ratio tPA/PAI-2.

For example, the kit may contain the necessary elements to quantify the expression levels of the ratio PAI-2/tPA.

In these cases, the patient may take the sample and perform the comparison using the kit at home.

If the quantification is performed by means of mRNA quantification, said kit may also comprise one or more primer sequences in order to detect and quantify the markers related to the activity of inflammation and/or proteolytic activity and/or plasminogen system.

If the quantification is performed by means of protein quantification, said kit may also comprise one or more substrates to detect and quantify the markers related to the plasminogen system, inflammation and/or proteolytic activity. Preferred substrates are antibodies, either monoclonal, polyclonal or fragments thereof. The kit may further comprise primary and secondary antibodies, and labeled antibodies.

In these cases, the kit may also comprise one or more calibration curves related to markers for bone resorption/remodeling in order to interpolate the expression level value and determine the bone loss rate. A kit for quantification of bone loss using biomarkers has been described previously in patent application GB1302257.9 by the same inventor.

The kit may be used and the use is not particularly limited, although the use in the method of the invention in any of its embodiments is preferred.

Optionally, the kit may comprise a data carrier such as for example in an electronic data carrier for saving the information regarding expression level of one or more regulated markers (e.g. obtained in step (a) of the method of the present invention) and/or saving the information regarding the state of peri-implant disease (e.g. obtained in step (b) of the method of the present invention).

“One or more” also as used herein includes one and the individualized specification of any number which is more than one, such as two, three, four, five, six, etc. “More than one” or “several” as used herein includes the individualized specification of any number which is more than one, such as two, three, four, five, six, etc.

Unless expressly specified otherwise, the term “comprising” is used in the context of this document to indicate that further members may optionally be present in addition to the members of the list introduced by “comprising”. It is, however, contemplated as a specific embodiment of the present invention that the term “comprising” encompasses the possibility of no further members being present, i.e. for the purpose of this embodiment “comprising” is to be understood as having the meaning of “consisting of”.

### EXAMPLES

### Example 1

### Gene Panel

### Subjects

This was a non-randomised, single-blinded (sample analysts) controlled clinical exploratory study which was approved by the local ethical committee, University of Göteborg, Sweden (Dnr: 652-10). The study included 25 subjects with healthy implant sites, 25 subjects with sites with peri-implant mucositis and 25 subjects with obvious clinical signs of peri-implantitis. The study was limited to a single evaluation time point. Study participants were selected from subjects previously rehabilitated with dental implants attending scheduled implant maintenance sessions at the Bränemark Clinic, Göteborg, Sweden. Each subject participated in the informed consent process and signed and dated the informed consent form (ICF) before any study related procedures were performed. One implant site per subject was evaluated, and the selected site was categorized as a healthy (HI), mucositis (MC) or peri-implantitis (PI) site on the basis of criteria described below. Peri-implant crevicular fluid (PICF) was collected from the implant sites using three pooled paper points per site. All persons involved in sample analysis and statistics were blinded to subject identity, and persons involved in sample analysis were also blinded to sample type (HI, MC or PI). Analysis of the expression of genetic markers was performed by an independent test laboratory (Tataa Biocenter, Göteborg, Sweden).

### Inclusion/Exclusion Criteria

For participation in the present study each subject fulfilled each of the general criteria 1-5 provided in Table 1. In order to be included in either the HI, MC or PI group, the subjects had to fulfill the inclusion criteria provided in Table 2, 3 and 4, respectively. The exclusion criteria for all three groups are provided in Table 5. Subject health conditions and treatments such as anti-inflammatory treatment, osteoporosis, diabetes, uncontrolled hyperparathyroidism, corticosteroid and bone anabolic therapies, history of malignancy, use of tobacco and/or other nicotine containing products were not exclusion criteria, but such conditions, treatments and use were recorded in the Case Report Forms (CRFs). All regular prescription medication and/or other regular treatment received within 30 days before subject enrolment, except anti-biotic treatment, was permitted and recorded in the CRFs. Antibiotic treatment within 3 months prior to study enrolment was prohibited.

Subject age, gender, oral health, Mombelli modified Bleeding Index (mBI), modified Plaque Index (mPI), Peri-Implant Pocket Depth (PIPD), height of attached mucosa and presence of suppuration was also recorded and quantified in the CRFs for all subjects.

Subject enrolment in both groups was performed in a consecutive manner provided the subjects fulfilled the eligibility criteria. The inclusion period was approximately one year, where subjects in all three groups were enrolled during the entire period.

Randomization was not applicable. The clinical investigator performing the clinical examination and PICF sampling was not blinded to the study parameters. All other persons involved in sample analyses were blinded to subject identity. The persons involved in performing qPCR analysis of PICF samples were blinded to the type of sample (HI, MC or PI). Persons involved in performing statistical analyses were not blinded to the study populations.

One subject from the healthy group was reported as screen failure due to antibiotic use, and thus, replaced by a new subject. All implants were placed during the period between 1985 and 2010. The main subject characteristics are described in Table 8. Mean time of function was 14 years, 13 years and 14 years, for the implants in the HI, MC and PI group, respectively. Nineteen (76%), 16 (64%) and 15 (60%) of the subjects in the HI, MC and PI group were provided with implants with machined surfaces (Bränemark System® implants), respectively. Eleven (44%), 9 (36%) and 8 (32%) subjects in the HI, MC and PI group were provided with implants with an anodically oxidized surface (Bränemark System® implants), respectively. Five subjects in the HI group and one subject each in the MC and PI group had implants with both types of surfaces. One subject in the MC group and 3 subjects in the PI group had implants with unidentified surfaces.

Implants were selected from both maxillae and mandibles, and no obvious differences with respect to implant placement and implant type, i.e. design, length, width, surface were observed between the three groups. No obvious clinical differences with respect to dental status and prosthetic construction between the groups were observed.

The bone loss and bone loss rate and average modified Mombelli Bleeding Index (mBI) and Plaque Index (mPI) fulfilled the inclusion criteria. Bone loss and bone loss rate for subjects in the peri-implantitis group, peri-implant probing depth (PIPD), average height of attached mucosa and number of subjects presenting suppuration are provided in Table 8. The data refer to the PICF sampling sites. All subjects in the HI group exhibited healthy mucosa, whereas the subjects in the MC and PI groups exhibited compromised mucosa health. Seven subjects (28%) exhibited suppuration at the test site in the MC group. PIPD was significantly different for subjects in the PI vs MC ((p<0.0001) PI vs HI (p<0.0001) and MC vs HI (p<0.0001) group, respectively.

General health condition data showed that more subjects in the MC and PI groups had compromised health conditions compared to the HI group. The subjects exhibited large differences in health conditions, where only one or two subjects in the study population exhibited a particular condition, such as rheumatoid arthritis, whereas several subjects exhibited the same condition, such as cardiovascular disease. Health conditions present in more than 3 subjects per study group in one or more groups are provided in Table 8. Subjects in the healthy group had significantly better oral hygiene compared to subjects in the mucositis (p<0.0001) and peri-implantitis group (P<0.0001). Smoking was significantly less common in the healthy group compared to subjects in the mucositis (P=0.0046) and peri-implantitis group (p=0.0001). History of periodontitis and peri-implantitis was significantly less frequent in the healthy compared to the peri-implantitis group, p=0.025 and p<0.0001, respectively. History of peri-implantitis was significantly more common in the peri-implantitis compared to the mucositis group (p=0.0023).

### Collection of Samples

The PICF sampling was performed as follows: Three sterile paper points (Roeke, Coltene, Germany) were inserted to the base of the peri-implant sulcus/pocket and left in situ for at least 60 seconds at the selected implant site. The three paper points were immediately transferred to one (1) 2 ml plastic tube (Microtube, 2 ml, Sarstedt, Numbrecht, Germany) containing RNALater preservation medium (Qiagen, Hilden, Germany); i.e. the three samples were pooled. Clean gloves were always used when handling the tubes. The paper points were completely immerged in the preservation medium. The pooled sample was transferred from the Bränemark Clinic the sampling day at ambient temperature to the local lab for analysis of gene markers.

### Handling and Analyses of Samples

Analysis of the qPCR samples were performed by TATAA Biocenter AB (Göteborg, Sweden) as per standard procedures, which has been previously described in Hall et al. (Eur J Oral Implant 2011, 4(4):371-382). In brief, RNA from cells attached to the paper points were extracted at TATAA Biocenter. The cells were then purified using Qiagen RNeasy Micro kit (Qiagen AB, Solna, Sweden) according to the manufacturer's instructions. Carrier RNA included in the kit was used to minimize losses of RNA during extraction. RNA was converted to cDNA using BioRad iScript cDNA synthesis kit (Bio-Rad Laboratories Inc., Hercules, Calif., USA) according to the manufacturer's instructions using 5 μl of the RNA. The cDNA was diluted to 50 μl in UltraPure water (Invitrogen Corp., Carlsbad, Calif., USA). Quantitative polymerase chain reaction (qPCR) assays of the samples were then performed. The analyzed biochemical markers are listed in the table 6. Perfecta SYBR Green Supermix (Quanta BioSciences, Gaithersburg, Md., USA) and 2 μl of cDNA template together with 0.4 μM of forward and reverse primer were used in the quantitative PCR. Each cDNA sample was quantified in duplicate. The following temperature protocol was employed: enzyme activation 3 min at 98° C. followed by 45 cycles of 20 seconds at 95° C., 20 seconds at 60° C. and 20 seconds at 72° C. Fluorescence detection was performed in a FAM/SYBR channel during the last temperature cycle. Experiments were performed on the LightCycler 480 System (Roche, Penzberg, Germany). After amplification a dissociation/melting curve was generated to verify that specific products were generated. Relative gene expression levels were calculated using the ΔΔCq method (Livak et al., Methods 2001, 25:402-408) using 90% efficiency for each assay and by normalizing gene expression of each gene by two reference genes (UBC and YWHAZ) that were selected using the freely available NormFinder17 program. The two genes were selected after running four genes, GAPDH, YWHAZ; UBC, HPRT-1, in the program. The selection of the four normalization genes was based on the results from our previous feasibility study (Hall et al., Eur J oral Implantol 2011, 4(4):371-382), where 9 reference genes were investigated and PICF sampling using paper points was also used. The main criterion was that the variation in reference gene expression should be minimal within and between the HI, MC and PI groups.

Limit Of Quantification (LOQ) was determined for Cq for all genes based on purified PCR product quantified by spectrophotometer. A five-point standard curve with four replicates in each point was generated for all assays, and run in ten-fold dilution series in concentrations between 10 and 10⁶ copies/μl. All data above the determined LOQ-values was omitted from the analyses. The procedure resulted in reduction of data scattering and narrowing of the data distributions, which increased the possibility for observation of significant differences in gene expression between the three subject groups.

In order to investigate if the qPCR analysis was inhibited by the sample matrix, e.g. presence of suppuration, 14 of the samples from the PI group were spiked with a known concentration of RNA-spike (#RS12JG, TATAA Biocenter AB) and compared by water samples spiked with the same concentration. One sample was taken with a sterile aspiration needle from one implant site exhibiting suppuration from the 14 subjects in the PI group. The aspiration needle sampling site was not the same but similar to the paper point sampling site. The aspiration needle (Metal Suction Tip, 0.7×70 mm 22 G, Mediplast, Malmö, Sweden) was inserted to the base of the peri-implant sulcus/pocket at the selected site. The needle containing the sample was immediately removed from the plastic syringe (1 ml, BD Plastipak, Mediplast, Malmo, Sweden) bent gently and put into one (1) 4.5 ml plastic cryo tube (Nunc CryoTube Vials, Fisher Scientific, Goteborg, Sweden). The lid of the tube was closed, and the tube was positioned and frozen at −196° C. in a thermos with liquid nitrogen and transported immediately to the lab (TATAA Biocenter) for inhibition analysis. Clean gloves were always used when handling the aspiration needles and the plastic tubes.

No significant difference could be observed between extraction with pure spike and sample matrix+spike. For all 14 tested samples the difference between pure spike and matrix+spike was Cq<0.3, which corresponds to less than 25% difference in gene expression, indicating that the sample matrix did not inhibit the reverse transcription or qPCR reaction. In addition, a secondary analysis showed that there were no significant differences in mean expression of TRAP and CatK when samples from subjects with bone loss rate <0.5 mm/year in the PI group were compared with the HI and MC subjects.

The expressions of the gene markers for the plasminogen system, inflammation and bone resorption in each group are provided in Table 9. The expressions of TRAP and CatK were not significantly different in any group.

### Statistical Methods for Examples 2 to 9

Differences in gene marker expression between the three study groups were estimated using analysis of variance (ANOVA). In the analysis of data, logarithmic data transformation was performed and ninety-five percent (95%) confidence intervals for differences between independent samples were used.

The normalized gene expression was calculated for each subject using the following expression after logarithmic transformation: normalized expression of gene g=(Cq(n)−Cq(g)) where Cq(g) is the number of amplification cycles for gene g, and Cq(n) is the normalization factor (mean number of amplification cycles for the selected reference genes) for the sample taken from the subject. The analysis of variances was performed using the normalized expression of gene g in the HI, MC and PI groups.

A p-value less than 0.05 would have been considered statistically significant if the investigation of possible differences between the HI, MC and PI groups comprised only two genetic markers. However, since the study comprised 8 markers after some had been excluded during the LOQ analysis, the Bonferroni correction for mass significance was used, and a p-value less than 0.0063 was considered statistically significant.

The calculated normalized gene expression for each subject was then correlated to the information on bone loss provided by the radiographs of that same subject at the same time point. Radiographic examination techniques are well known in the field and can be performed as described in Ahlqvist et al. (Int J Oral Maxillofac Implants 1990, 5(2):155-163). The bone level can be measured on the radiographs and it is defined as the distance from the junction between the fixture and its abutment to the crest of the marginal bone mesially and distally to the implants.

### Statistical Methods for Examples 10 and 11

The gene markers were treated as continuous variables and presented as mean, standard deviation (SD), median, minimum and maximum. For comparison between two groups Fisher's exact test was used for dichotomous variables. The correlation analysis was performed with Spearman's correlation rank test. In order to identify gene markers that were different pairwise between the three groups, univariable logistic regression analysis was performed. In order to select independent gene markers all markers with a univariable p<0.15 were included into a stepwise multiple regression analysis. The results from the logistic regression analyses are given as unadjusted and adjusted Odds Ratio (OR) with 95% confidence interval and p-values. OR is the change in Odds of belonging to the P group for one unit increase in the gene expression for a given marker. The probability to belong to the P group as a function of the independent gene marker is calculated from the logistic regression and given in graphs. As goodness fit for using a selected marker to distinguish between pairwise pen-implant tissue conditions (PI, MC, HI) the ROC-curve was determined. The area under the ROC curve is the probability that a randomly selected subject that has peri-implantitis will have up- or down regulation of the selected marker compared with a randomly selected subject that does not have peri-implantitis. If the area under the ROC curve is 0.5, it means that the given marker cannot be used at all to distinguish between peri-implantitis and the other two peri-implant tissue conditions studied (HI, MC). An area under the ROC curve equal to 1 means that the marker can be used to perfectly distinguish between the PI, MC and HI conditions, sensitivity=1.0 and specificity=1.0. Due to the very strong negative correlation between tPA and ratio tPA/PAI-2 (rs=−0.97, p<0.0001), the ratio was only included in the univariable logistic analyses and not in the multiple logistic analyses.

The freely available program Normfinder30 was used to identify the best reference genes for data normalization. The calculations resulted in selection of a combination of two genes with equal weight, YWHAZ and UBC, for normalization of data, since the variation in reference gene expression was minimal within and between the HI, MC and PI groups when the mean of both genes were used. The normalized gene expression was calculated for each subject using the following expression after logarithmic transformation: normalized expression of gene g=Cq(n)−Cq(g) where Cq(g) is the number of amplification cycles for gene g, and Cq(n)=(Cq(YWHAZ)+Cq(UBC))/2 is the normalization factor for the sample taken from the subject. The statistical analyses were performed using the normalized expression of gene g in the HI, MC and PI groups.

TABLE 1
General Inclusion Criteria

given informed consent to participate in the study
18 years or older
rehabilitated with dental implant in the maxilla and/or mandible. The
implants should have been in function for more than 1 year
conditions that allow for collection of PICF using periodontal paper
points and aspiration needles
at least two evaluable radiographs of the three implants taken at two
different time points after at least 1 year in function must be available.
The most recent radiographs shall not be older than 3 months

TABLE 2
Inclusion Criteria for Subjects in the HI group

no radiographic evidence of pathologic bone loss (rate of bone loss not
exceeding 1.5 mm first year of implant loading and 0.2 mm/year
thereafter) around any of the implants
no signs of inflammation and no/limited bleeding on superficial probing
around any of the implants (modified bleeding index, mBI = 0 or 1)
no suppuration on palpation at any of the implants

TABLE 3
Inclusion Criteria for Subjects in the MC group

no radiographic evidence of pathologic bone loss (rate of bone loss not
exceeding 1.5 mm first year of implant loading and 0.2 mm/year
thereafter) at any of the implant sites
bleeding on superficial probing around at least three implants (modified
bleeding index, mBI = 2 or 3)
redness and swelling of the peri-implant mucosa around implants
presenting bleeding on superficial probing

TABLE 4
Inclusion criteria for subjects in the PI group

at least three implant sites with radiographs showing obvious signs of
pathologic bone loss
bleeding on superficial probing around at least three implants (modified
bleeding index, mBI = 2 or 3)
suppuration upon palpation around implants presenting radiographic bone
loss and bleeding on superficial probing

TABLE 5
Exclusion Criteria

not able to give his/her informed consent to participate in the study
history of antibiotic treatment within 3 months prior to study inclusion
has had augmentation procedures performed at any of the selected implant
sites
has implant supported overdenture in the jaw of interest

TABLE 6
Analysed gene markers

| | | SEQ | Abbrevia- | Main biological |
|---|---|---|---|---|
| # | Gene marker | ID NO | tion | process |
| 1 | Interleukin-1β | 13 | IL-1b | Inflammation |
| | | | (IL-1β) | |
| 2 | Interleukin-8 | 4 | IL-8 | Inflammation |
| 3 | Tissue inhibitor matrix | 5 | TIMP-1 | Inhibitor of |
| | metalloproteinase-1 | | | proteolytic activity |
| 4 | Plasminogen activator | 6 | PAI-2 | Inhibitor of |
| | inhibitor-2 | | | proteolytic activity |
| 5 | Interleukin 6 | 2 | IL-6 | Bone degradation |
| 6 | Tyrosine 3/tryptophan | 14 | YWHAZ | Normalisation gene |
| | 5-monoxygenase activa- | | | |
| | tion protein, zeta poly- | | | |
| | peptide | | | |
| 7 | Ubiquitin C | 15 | UBC | Normalisation gene |

TABLE 7
Soft tissue status

| | HI Group | MC Group | PI Group |
|---|---|---|---|
| PIPD (mm) | 2.1 (SD = 0.7) | 3.1 (SD = 0.9) | 5.5 (SD = 2.3) |
| Height of attached | 1.3 (SD = 1.0) | 1.4 (SD = 1.1) | 1.3 (SD 1.2) |
| mucosa (mm) | | | |
| Suppuration | 0 | 7 | 25 |
| (#subjects) | | | |

TABLE 8
Subject status for the three studied groups

| | Peri-implantitis Group (PI) | Mucositis Group (MC) | Healthy Group (HI) |
|---|---|---|---|
| | (n = 25) | (n = 25) | (n = 25) |
| Mean age | 68 (12) | 71 (9) | 72 (15) |
| | 44 (50; 89) | 39 (55; 95) | 33 (29; 95) |
| Gender | 17 F (68%) | 6 F (24%) | 15 F (60%) |
| | 8 M (32%) | 19 M (76%) | 10 M (40%) |
| Implants: mean time | 14 (7) | 13 (7) | 14 (7) |
| in function | 17 (4; 25) | 13 (2; 26) | 13 (1; 24) |
| PIPD (mm) | 5.5 (2.3) | 3.1 (0.9) | 2.1 (0.7) |
| | 5 (3; 10.5) | 3 (2; 5) | 2 (1; 4) |
| | P vs M; p < 0.0001 | M vs H: p < 0.0001 | |
| | P vs H: p < 0.0001 | | |
| Average marginal | 5.1 mm (2.3 mm); 4.4 | bone loss not | bone loss not |
| bone loss at qPCR | (1.2; 10.5) | exceeding 1.5 mm | exceeding 1.5 mm |
| sample site | and | first year of implant | first year of implant |
| | 0.7 mm/year (1.1); 0.5 | loading and 0.2 | loading and 0.2 |
| | (0; 3.3) | mm/year thereafter | mm/year thereafter |
| History of | 11 (44%) | 9 (36%) | 3 (12%) |
| periodontitis | P vs H; p = 0.025 | | |
| History of peri- | 14 (45%) | 3 (12%) | 0 |
| implantitis | PI vs MC; p = 0.0023 | | |
| | PI vs HI; p < 0.0001 | | |
| Smoker | 14 (45%) | 10 (40%) | 1 (4%) |
| | PI vs HI; p = 0.0001 | MC vs HI; | |
| | | p = 0.0046 | |
| Poor oral hygiene | 12 (48%) | 13 (52%) | 0 |
| | PI vs HI; p < 0.0001 | MC vs HI; p < 0.0001 | |
| High blood pressure | 8 (32%) | 8 (32%) | 7 (28%) |
| Cardiovascular | 6 (24%) | 6 (24%) | 4 (16%) |
| disease | | | |
| High cholesterol | 4 (16%) | 2 (8%) | 2 (8%) |
| Allergy | 5 (20%) | 4 (16%) | 4 (16%) |

Mean (SD)/Median(Min; Max) and p < 0.05 reported

TABLE 9
Gene Expression in samples from subjects with peri-implantitis, mucositis and healthy
subjects (Logarithmic transformation of normalised Cq-values as described in section
“Statistical Methods for Examples 2 to 9”; n = number of subjects)

| | Peri-implantitis | Mucositis | Healthy |
|---|---|---|---|
| Variable | (n = 25) | (n = 25) | (n = 25) |
| Il1β | 3.99 (0.67) | 2.84 (1.31) | 3.09 (1.15) |
| | 4.11 (1.63; 4.91) | 2.84 (−0.37; 4.70) | 3.41 (0.06; 4.69) |
| | n = 20 | n = 23 | n = 25 |
| IL-8 | 4.70 (0.98) | 3.53 (1.38) | 3.86 (1.35) |
| | 4.65 (2.25; 6.24) | 3.37 (0.49; 6.14) | 4.02 (0.80; 6.15) |
| | n = 20 | n = 23 | n = 25 |
| PAI-2 | −4.20 (1.17) | −3.16 (0.90) | −3.65 (1.09) |
| | −4.02 (−6.26; −1.61) | −3.15 (−4.71; −1.38) | −3.62 (−6.27; −1.73) |
| | n = 19 | n = 22 | n = 24 |
| tPA | −2.47 (2.69) | 0.339 (1.799) | −1.40 (2.06) |
| | −3.06 (−7.08; 3.21) | 1.080 (−3.905; 2.973) | −1.03 (−6.26; 1.37) |
| | n = 16 | n = 18 | n = 20 |
| TRAP | −7.26 (2.11) | −7.02 (0.81) | −6.87 (1.37) |
| | −7.80 (−9.49; −2.14) | −7.13 (−8.68; −5.96) | −6.66 (−8.63; −4.21) |
| | n = 14 | n = 11 | n = 15 |
| CatK | −5.64 (1.86) | −5.52 (1.52) | −5.29 (1.92) |
| | −6.14 (−7.92; −0.75) | −5.84 (−7.50; −2.85) | −5.37 (−8.02; −1.58) |
| | n = 17 | n = 17 | n = 18 |
| tPA/PAI-2 | 0.520 (0.752) | −0.137 (0.670) | 0.412 (0.664) |
| | 0.628 (−1.332; 1.944) | −0.387 (−1.024; 1.356) | 0.250 (−0.352; 2.658) |
| | n = 16 | n = 17 | n = 20 |

For continuous variables Mean (SD)/Median (Min; Max)/n = is presented.

### Example 2

Levels of IL-1β and PAI-2 are quantified in peri-implant crevicular fluid obtained from a patient that has undergone treatment for peri-implantitis. Expression levels of IL-1β and PAI-2 are quantified by qPCR as described in Example 1. The IL-1β value obtained is then compared with the expression levels of PAI-2 before and after treatment in the same sample taken from the same patient. See FIG. 1. The treatment has had an effect since the expression levels of IL-1β and PAI-2 have been reversed by the treatment.

### Example 3

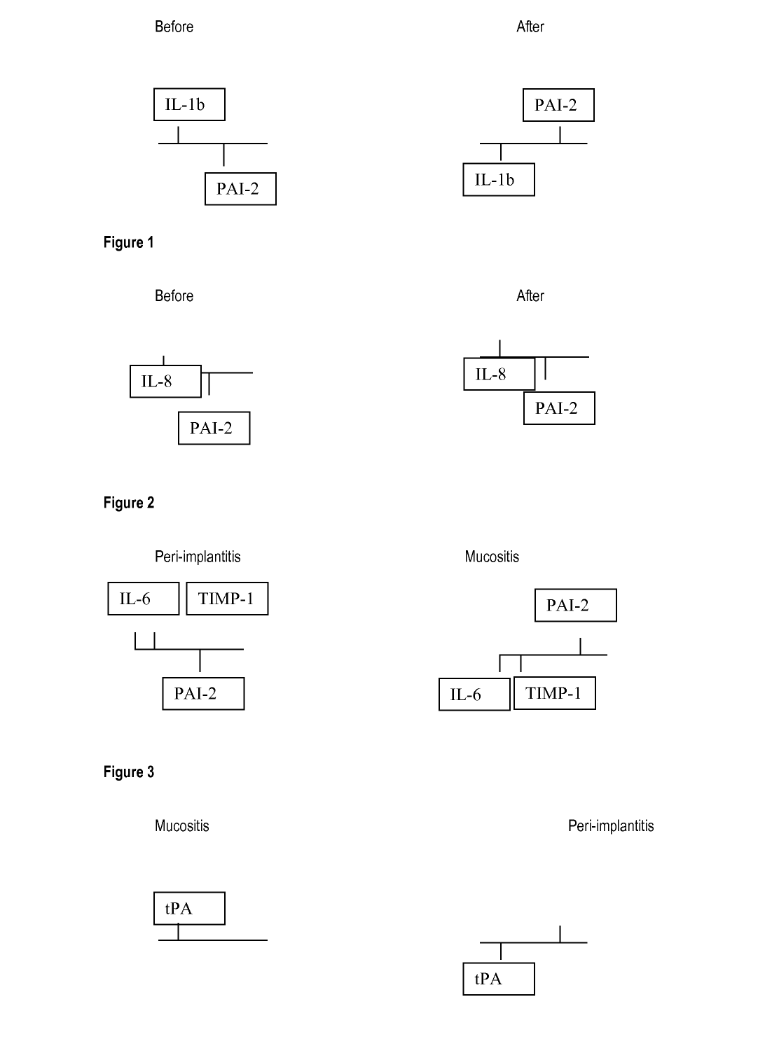

Expression levels of IL-8 and expression levels of PAI-2 are quantified in peri-implant crevicular fluid obtained from a patient that has undergone treatment for peri-implantitis. Expression levels of IL-8 and PAI-2 are quantified by qPCR as described in example 1. The treatment has had no effect since the expression levels of IL-8 and PAI-2 have not been reversed by the treatment. See FIG. 2.

### Example 4

Levels of IL-6, TIMP-1 and PAI-2 are quantified in peri-implant crevicular fluid obtained from a patient that has undergone treatment for peri-implantitis. Expression levels of IL-6, TIMP-1 and PAI-2 are quantified by qPCR as described in example 1. The treatment has had an effect since the expression levels of IL-6, TIMP-1 and PAI-2 have been reversed by the treatment. See FIG. 3.

### Example 5

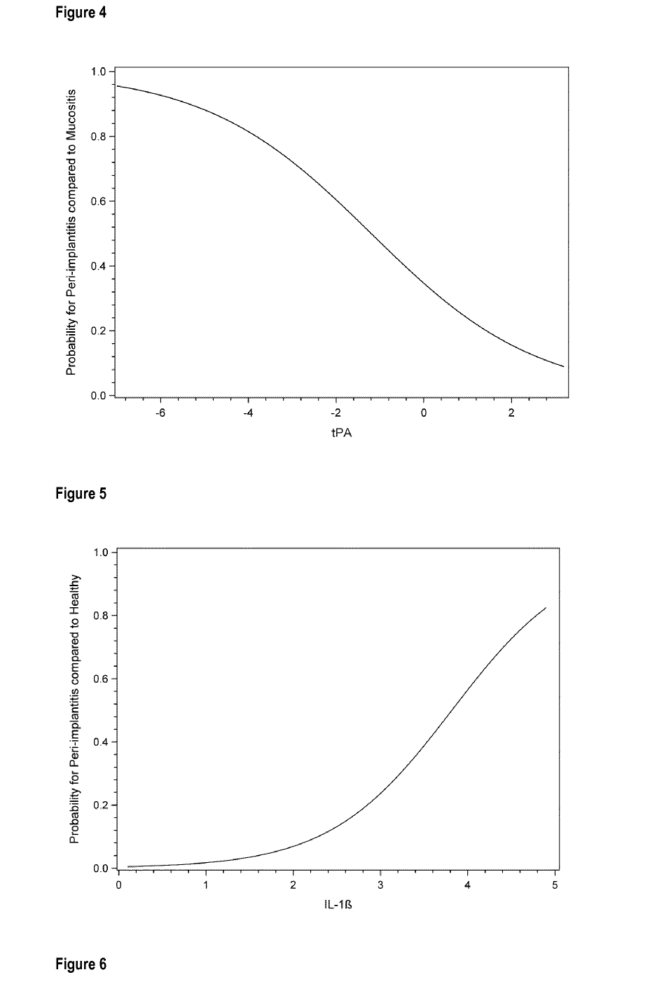

Levels of tPA are quantified in peri-implant crevicular fluid obtained from a patient that has undergone treatment for mucositis. Expression levels of tPA are quantified by qPCR as described in example 1. The treatment has had an effect since the level of tPA has been reduced by a factor of about 3. See FIG. 4.

### Example 6

In one or more of the Examples 2, 4 and 5 the clinician may provide the subject with a standard program of care oral hygiene treatment. A follow up visit may be scheduled within several months or a year.

### Example 7

In Example 3, the clinician concludes that the treatment did not have an effect, provides the subject with additional oral hygiene treatment and schedule a follow up visit within a few months. If the markers have not changed at the second follow up visit, the clinician may decide to perform surgical treatment of the site.

### Example 8

In one or more of the Examples 2, 4 and 5, a subject exhibits obvious signs of peri-implantitis, i.e. peri-implant inflammation, swelling, redness, suppuration and pathologic, crater shaped marginal bone loss. However, the markers were changed by the non-surgical treatment, and the clinician concluded that surgical treatment was unnecessary and provided the subject with additional oral hygiene treatment, maintenance protocol and scheduled a follow up visit within a few months.

### Example 9

In another example, a sample taken from a subject exhibiting inflammation and bleeding on probing shows that the ratio between IL-1β and PAI-2 is less than 2. The clinician concludes that proteolytic activity is likely very low and insignificant, and provides the subject with standard of care oral hygiene program. A follow up visit is scheduled within several months or a year.

It was not necessary to expose the subjects of examples 1 to 9 to radiation, and the clinician did not have to wait until the bone degradation had progressed to a measurable level assessed by radiographs in the examples in order to determine the state of peri-implant disease.

### Example 10

Significant markers from the univariable logistic regression to discriminate between the PI and MC groups were high values of IL-1β (p=0.0065), high values of IL-8 (p=0.0082), low values of PAI-2 (p=0.0089) and low values of tPA (p=0.0054), See Table 10 for Odds ratio with 95% confidence interval and area under the ROC-curve (AUC). After tPA was entered into the stepwise analysis no other marker was entered into the model. The area under ROC-curve (AUC) was 0.81. The probability of belonging to the PI compared to MC group as a function of increasing tPA is provided in FIG. 5.

TABLE 10
Explanatory variables to samples from subjects with mucositis and peri-implantitis compared to
samples from healthy subjects, and samples from subjects with peri-implantitis compared to samples from
subjects with mucositis

| | Mucositis vs. Healthy | | | Peri-Implantitis vs. Healthy | | | Peri-Implantitis vs. Mucositis | | |
|---|---|---|---|---|---|---|---|---|---|
| | OR (95% CI) | | | OR (95% CI) | | | OR (95% CI) | | |
| Variable | Group | p-value | AUC | Group | p-value | AUC | Group | p-value | AUC |
| IL1-β | 0.84 (0.52-1.35) | 0.48 | 0.55 | 4.19 (1.32-13.32) | 0.015 | 0.81 | 3.48 (1.42-8.52) | 0.0065 | 0.78 |
| IL-8 | 0.83 (0.54-1.28) | 0.40 | 0.59 | 1.93 (1.04-3.55) | 0.036 | 0.68 | 2.31 (1.24-4.30) | 0.0082 | 0.74 |
| PAI-2 | 1.67 (0.88-3.16) | 0.11 | 0.62 | 0.64 (0.36-1.13) | 0.13 | 0.64 | 0.37 (0.17-0.78) | 0.0089 | 0.76 |
| tPA | 1.70 (1.08-2.68) | 0.021 | 0.74 | 0.82 (0.61-1.10) | 0.18 | 0.63 | 0.59 (0.41-0.86) | 0.0054 | 0.81 |
| TRAP | 0.89 (0.44-1.78) | 0.73 | 0.56 | 0.87 (0.56-1.35) | 0.54 | 0.60 | 0.91 (0.56-1.49) | 0.71 | 0.64 |
| CatK | 0.92 (0.62-1.37) | 0.69 | 0.51 | 0.90 (0.63-1.30) | 0.58 | 0.56 | 0.96 (0.64-1.44) | 0.84 | 0.55 |
| tPA/PAI-2 | 0.22 (0.06-0.86) | 0.029 | 0.73 | 1.26 (0.48-3.31) | 0.64 | 0.61 | 3.79 (1.21-11.89) | 0.022 | 0.76 |

All tests were performed with univariable logistic regression

Significant markers from the univariable logistic regression to discriminate between subjects with peri-implantitis and subjects in the HI group were high values of IL-1β (p=0.015), and high values of IL-8 (p=0.036). See Table 10. After IL-1β was entered into the stepwise analysis no other marker was entered into the model. Area under ROC-curve was 0.81. The probability of belonging to the PI compared to HI group as a function of increasing IL-1β is provided in FIG. 6.

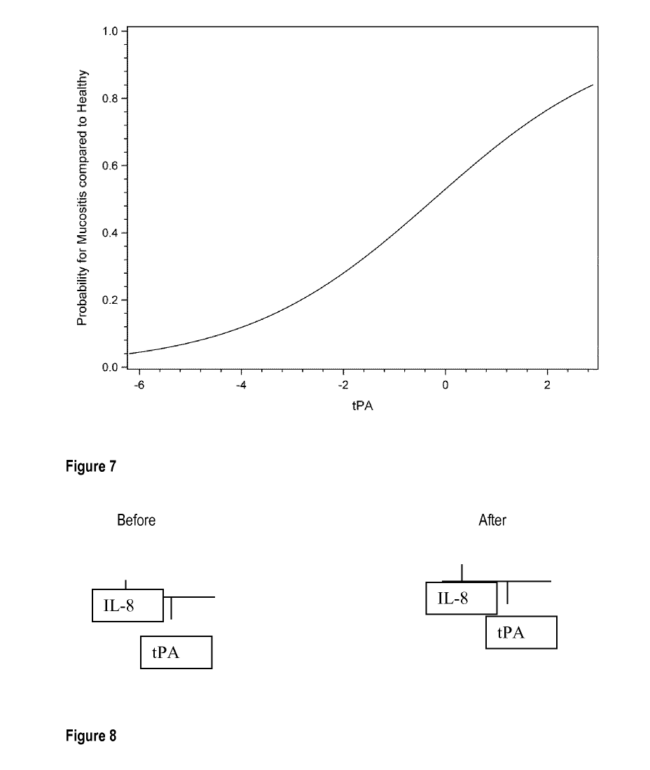

Significant marker from the univariable logistic regression to discriminate between the MC group and the HI group were high values of tPA (p=0.021). See Table 10. The probability to belong to the MC group compared with the HI group as a function of increasing tPA is shown in FIG. 7.

The ratio tPA/PAI-2 was significantly lower in the MC group compared with the HI group (p=0.029) and the PI group (0.022). See Table 10. The tPA/PAI-2 ratio had a very strong negative correlation with tPA (rs=−0.97, p<0.0001).

IL-1β and IL-8 were up-regulated and tPA and PAI-2 were down-regulated in samples from the subjects in the PI group compared with the MC group, and tPA was up-regulated in the MC group compared with the HI group. The tPA/PAI-2 ratio was significantly down-regulated in samples from the MC group compared with samples from the HI and PI groups.

The data suggests that mucositis and peri-implantitis are two different conditions separated by the host inflammatory response to proteolytic activity. Biofilms will always be present at implants and there will always be an inflammatory host response, which is balanced between tissue breakdown and repair in healthy subjects and subjects with mucositis, whereas the balance is shifted pathologically towards tissue destruction in subjects with peri-implantitis. The results from this study indicate that the plasminogen system plays an important role in such a balance.

Except for a history of peri-implantitis, the subject health conditions did not seem to influence the inflammatory and plasminogen system response in subjects with mucositis and peri-implantitis. See Table 8. Thus, except for a history of peri-implantitis, subject health conditions did not contribute to differences between mucositis and peri-implantitis in this study.

Poor oral hygiene and the modified plaque index were similar for subjects in the MC and PI groups. Therefore, it seems that a successful treatment of mucositis and peri-implantitis may not depend on the quantity of biofilm removed from the site, but rather the removal or inhibition of the microbial activity within the biofilm.

In addition to removal of the biofilm at the infected site, and possible bone augmentation procedures, surgical treatment of peri-implantitis aim at pocket elimination to enhance oral hygiene measures The outcome, though, is somewhat unpredictable and not seldom bleeding on probing and presence of pus may appear after a short period, as based on own experiences. It would be of value to know, whether this is an expression of a continuous active soft tissue degradation and bone resorption requiring further surgery or an infection and inflammation not correlated to ongoing significant tissue degradation and bone resorption. Should the disease progression be in a phase with infection but without ongoing significant tissue degradation and bone resorption, it may be reasonable to question the use of surgical interventions. This is perhaps in contrast to treating the periodontitis patient, in whom the outcome in general is predictable and successful, and where the awareness of the fact that this disease displays various phases of activity has not changed the clinical handling.

Furthermore, it may be of value to be able to rapidly assess the efficacy of a treatment for peri-implantitis. Since radiographs may be used to assess bone level changes larger than 0.5 mm (Ahlqvist J, Borg K, Gunne J, Nilson H, Olsson M, Astrand P. Osseointegrated implants in edentulous jaws: a 2-year longitudinal study. Int J Oral Maxillofac Implants 1990; 5(2): 155-163.), long time periods may be required before the bone loss has reached such a level. If unambiguous diagnosis of mucositis and peri-implantitis independent of radiographs can be established, it may constitute a powerful tool for rapid assessment of the peri-implant tissue condition and effect of the treatment.

The different gene panels for the subjects with mucositis and peri-implantitis in this study suggest that markers for the plasminogen system and inflammation could provide such a powerful tool for rapid establishment of ongoing tissue and bone degradation. The area under the ROC curve was 0.81 for tPA when subjects in the P group were compared with subjects in the M group. Thus, the probability was 81% that a randomly selected subject that had peri-implantitis would have a lower value of tPA when compared to a randomly selected subject that had mucositis, (see Table 10). Up-regulation of tPA was a strong predictor of mucositis in our study.

### CONCLUSIONS

Presence of suppuration did not to inhibit the qPCR analysis and a barrier for migration of cells expressing bone resorption markers did not seem to be present at the sampling sites of the subjects in the PI group. The markers IL-1β and IL-8 were significantly higher in the PI group and tPA and PAI-2 was significantly lower for the PI group compared with the MC group. tPA was the strongest marker and could be used to distinguish between mucositis and peri-implantitis irrespective of the bone loss rate. The results suggested that the plasminogen system was sufficiently up-regulated to allow for resolution of inflammation and healing at the inflamed implant site in subjects with mucositis, whereas such up-regulation was insufficient resulting in impaired healing and prolonged inflammation in subjects with peri-implantitis.

### Example 11

Expression levels of IL-8 and expression levels of tPA are quantified in peri-implant crevicular fluid obtained from a patient that has undergone treatment for peri-implantitis. Expression levels of IL-8 and tPA are quantified by qPCR as described in Example 1. The treatment has had no effect since the expression levels of IL-8 and tPA have not been reversed by the treatment. See FIG. 8.

Sequence list (Amplicon context sequences, Primer Sequence Disclosure: A Clarification of the MIQE Guidelines. Bustin et al., Clin Chem, 2011 16295).

| | |
|---|---|
| Interleukin-1beta (IL-1β): SEQ ID NO 1 | Interleukin-6 (IL-6): SEQ ID NO 2 |
| Matrix metalloproteinase-8 (MMP8) SEQ | Tartrate resistant acid phosphate (TRAP): |
| ID NO 3 | SEQ ID NO 4 |
| Cathepsin K: SEQ ID NO 5 | Osteoprotegerin (OPG): SEQ ID NO 6 |
| Receptor activator of the NF-kB ligand | Interleukin-8 (IL-8): SEQ ID NO 8 |
| (RANKL): SEQ ID NO 7 | |
| Homo sapiens dickkopf 1 homolog | Tissue inhibitor of matrix metalloproteinase |
| (Xenopus laevis) (DKK1): SEQ ID NO 9 | (TIMP-1): SEQ ID NO 10 |
| Tissue plasminogen activator (tPA): | Plasminogen activator inhibitor type 2 |
| SEQ ID NO 11 | (serpinB2) (PAI-2): SEQ ID NO 12 |
| Osteocalcin (PMF1 or OC): SEQ ID NO 13 | YWHAZ (Reference gene): SEQ ID NO 14 |
| UBC (Reference gene): SEQ ID NO 15 | |

SEQ ID NO 1

| | |
|---|---|
| Gene: | Interleukin-1beta (IL-1b/IL-β) |
| Accession number: | NM_000576.2 |

Amplicon context sequence:
ctccaggagc ccagctatga actccttctc cacaagcgcc
ttcggtccag ttgccttctc cctggggctg ctcctggtgt
tgcctgctgc cttccctgcc ccagtacccc caggagaaga
ttccaaagat gtagccgccc cacacagaca gccactcacc
tcttcagaac gaattgacaa
SEQ ID NO 2

| | |
|---|---|
| Gene: | Interleukin-6 (IL-6) |
| Accession number: | NM_000600.3 |

Amplicon context sequence:
attcaatgag gagacttgcc tggtgaaaat catcactggt
cttttggagt ttgaggtata cctagagtac ctccagaaca
gatttgagag tagtgaggaa caagccagag ctgtgcagat
gagtacaaaa gtcctgatcc agttcctgca gaaaaaggca
aagaatctag atgcaataac cacccctgac ccaaccacaa
atgccagcct gctgacgaag
SEQ ID NO 3

| | |
|---|---|
| Gene: | Matrix metalloproteinase-8 (MMP8) |
| Accession number: | NM_002424.2 |

Amplicon context sequence:
ggtgacaatt ctccatttga tggacccaat ggaatccttg
ctcatgcctt tcagccaggc caaggtattg gaggagatgc
tcattttgat gccgaagaaa catggaccaa cacctccgca
aattacaact tgtttcttgt tgctgctcat gaatttggcc
attctttggg gctcgctcac tcctctgacc ctggtgcctt
gatgtatccc aactatgctt tcagggaaac cagcaactac
tcactccctc
SEQ ID NO 8

| | |
|---|---|
| Gene: | Interleukin-8 (IL-8) |
| Accession number: | NM_000584.3 |

Amplicon context sequence:
ggcacaaact ttcagagaca gcagagcaca caagcttcta
ggacaagagc caggaagaaa ccaccggaag gaaccatctc
actgtgtgta aacatgactt ccaagctggc cgtggctctc
ttggcagcct tcctgatttc tgcagctctg tgtgaaggtg
cagttttgcc aaggagtgct aaagaactta gatgtcagtg
cataaagaca tactccaaac ctttccaccc caaatttatc
SEQ ID NO 10

| | |
|---|---|
| Gene: | Tissue inhibitor of matrix |
| | metalloproteinase (TIMP-1) |
| Accession number: | NM_003254.2 |

Amplicon context sequence:
gagagacacc agagaaccca ccatggcccc ctttgagccc
ctggcttctg gcatcctgtt gttgctgtgg ctgatagccc
ccagcagggc ctgcacctgt gtcccacccc acccacagac
ggccttctgc aattccgacc tcgtcatcag ggccaagttc
gtggggacac cagaagtcaa ccagaccacc ttataccagc
SEQ ID NO 11

| | |
|---|---|
| Gene: | Tissue plasminogen activator |
| | (tPA) |
| Accession number: | NM_000930.3 |

Amplicon context sequence:
acttaaagga ggccggagct gtggggagct cagagctgag
atcctacagg agtccagggc tggagagaaa acctctgcga
ggaaagggaa ggagcaagcc gtgaatttaa gggacgctgt
gaagcaatca tggatgcaat gaagagaggg ctctgctgtg
tgctgctgct gtgtggagca gtcttcgttt cgcccagcca
ggaaatccat gcccgattca gaagaggagc
SEQ ID NO 12

| | |
|---|---|
| Gene: | Plasminogen activator inhibitor |
| | type 2 (serpinB2)(PAI-2) |
| Accession number: | NM_001143818.1 |

Amplicon context sequence:
gccccaccca gaacctcttc ctctccccat ggagcatctc
gtccaccatg gccatggtct acatgggctc caggggcagc
accgaagacc agatggccaa ggtgcttcag tttaatgaag
tgggagccaa tgcagttacc cccatgactc cagagaactt
taccagctgt gggttcatgc
SEQ ID NO 14

| | |
|---|---|
| Gene: | YWHAZ (Reference gene) |
| Accession number: | NM_001135702.1 |

Amplicon context sequence:
aatcttctct cagttgctta taaaaatgtt gtaggagccc
gtaggtcatc ttggagggtc gtctcaagta ttgaacaaaa
gacggaaggt gctgagaaaa aacagcagat ggctcgagaa
tacagagaga aaattgagac ggagctaaga gatatctgca
atgatgtact gtctcttttg gaaaagttct tgatccccaa
tgcttcacaa gcagagagca aagtcttcta tttgaaaatg
aaaggagatt actaccgtta cttggctgag gttgccgctg
gtgatgacaa gaaagggatt gtcgatcagt cacaacaagc
ataccaagaa gcttttgaaa tcagcaaaaa ggaaatgcaa
ccaacacatc ctatcagact gggtctggcc cttaacttct
ctgtgttcta ttatgagatt
SEQ ID NO 15

| | |
|---|---|
| Gene: | UBC (Reference gene) |
| Accession number: | NM_001135702.1 |

Amplicon context sequence:
ttggagggtc gtctcaagta ttgaacaaaa gacggaaggt
gctgagaaaa aacagcagat ggctcgagaa tacagagaga
aaattgagac ggagctaaga gatatctgca atgatgtact
gtctcttttg gaaaagttct tgatccccaa tgcttcacaa
gcagagagca aagtcttcta tttgaaaatg aaaggagatt
actaccgtta cttggctgag gttgccgctg gtgatgacaa
gaaagggatt gtcgatcagt cacaacaagc ataccaagaa
gcttttgaaa tcagcaaaaa ggaaatgcaa ccaacacatc
ctatcagact gggtctggcc cttaacttct

### ITEMS OF THE PRESENT INVENTION

1. A method for measuring the state of peri-implant or periodontal disease, wherein the method comprises the steps of:
   a) quantifying the expression level of one or more regulated markers of a group of markers forming a panel, said one or more regulated markers being related to inflammation and/or proteolytic activity or ratio thereof in an ex vivo sample; and
   b) determining the state of peri-implant disease by comparing the expression level obtained in step a with a reference value.
2. The method according to item 1, wherein said panel comprises a marker selected from a group of proteinase inhibitors.
3. The method according to one or more of the preceding items, wherein said marker is selected from IL-1β, IL-4. IL-6, IL-8, IL-12, IL18, MMP-8, MMP-9, TIMP-1, tPA, PAI-2, or any combination thereof.
4. The method according to item 1, wherein said panel comprises at least the following markers IL-1β, IL-4, IL-8, IL-12, TIMP-1, PAI-2, or any combination thereof.
5. The method according to one or more of the preceding items, wherein the ex vivo sample is a body fluid or tissue.
6. The method according to item 5, wherein said fluid is an oral fluid, and/or blood, and/or serum, and/or plasma, and/or saliva.
7. The method according to items 5 or 6, wherein said fluid is gingival crevicular fluid.
8. The method according to one or more of items 5 to 7, wherein said fluid is peri-implant crevicular fluid.
9. The method according to item 5, wherein said tissue is bone and/or connective tissue, and/or gingiva, and/or mucosa.
10. The method according to item 5 and/or 9, wherein said tissue is bone, and/or implant supporting tissue, and/or bone adjacent to an implant and/or bone adjacent to a tooth.
11. The method according to any one of the preceding items comprising the step of, using a reference value from an ex vivo sample taken from the same source at a different period in time to analyze if markers have been up- or down regulated in said panel.
12. The method according to any one of the preceding items comprising the step of, using a reference value having markers indicating a history of periodontitis to analyze if markers have been up- or down regulated in said panel.
13. The method according to any one of the preceding items comprising the step of, using a reference value having markers indicating a history of periimplantatis to analyze if markers have been up- or down regulated in said panel.
14. The method according to one or more of the preceding items, wherein said quantification is performed by means of nucleic acid quantification.
15. The method according to item 14, wherein said quantification is performed by means of qPCR, and/or by means of Northern Blot.
16. The method according to one or more of items 1 to 12, wherein said quantification is performed by means of protein quantification.
17. The method according to item 16, wherein said quantification is performed by means of an immunoassay, and/or by means of ELISA, and/or by means of a radioimmunoassay, and/or by means of a magnetic immunoassay, and/or by means of a fluorescent immunoassay, and/or by means of immunoprecipitation, and/or by means of surface plasmon resonance, and/or by means of immunohistochemistry, and/or by means of Western Blot, or any combination thereof.
18. The method according to one or more of the preceding items, wherein the determined expression level is indicative of the presence or absence of a condition that affects bone.
19. The method according to one or more of the preceding items, wherein the determined expression level is indicative of the presence or absence of a condition that affects mucosa, gingival, bone supporting implants and/or teeth.
20. The method according to one or more of the preceding items, wherein the sample is obtained from a patient who has one or more implants.
21. The method according to item 20, wherein the implant is a bone anchored implant.
22. The method according to items 20 and/or 21, wherein the implant is a dental implant, and/or a hip implant, and/or a knee implant or a combination thereof.
23. A kit for carrying out the method according to one or more of the preceding items.
24. The kit according to item 23, wherein the kit further comprises a sample collecting device.
25. The kit according to item 24, wherein the sample collecting device is a sterile paper point and/or a syringe and/or a biopsy device.
26. The kit according to one or more of items 23 to 25, wherein the kit further comprises a preservation medium for preserving the sample.
27. The kit according to one or more of items 23 to 26, wherein the kit further contains a box to send the sample to a central laboratory.
28. The kit according to one or more of items 23 to 27, wherein the kit further contains the necessary elements to quantify the expression levels of one or more markers related to inflammation and/or proteolytic activity or combination thereof.

1. A method for determining the state of peri-implant disease, wherein the method comprises the steps of:
a) quantifying the expression level of one or more regulated markers selected from the group consisting of tPA, IL-4, IL-6, IL-10, IL-12, IL18, TIMP-1 and PAI-2, or any combination thereof, or any ratio thereof; and/or quantifying the expression levels of one or more of the following combinations of markers: tPA and tPA/PAI-2; PAI-2 and tPA/PAI-2; tPA, PAI-2 and tPA/PAI-2; tPA and IL-1β; tPA and TIMP-1; IL-1β and IL-8; IL-8 and PAI-2; IL-6, TIMP-1 and PAI-2; tPA and PAI-2; IL-1β, IL-8 and IL-6; IL-1β, IL-8, IL-6 and TIMP-1; IL-1β, IL-8, IL-6, TIMP-1 and tPA; IL-1β, IL-8, IL-6, TIMP-1 and PAI-2; IL-1β, IL-8, PAI-2 and tPA; IL-1β, IL-8, and PAI-2; IL-1β, IL-8 and tPA; IL-1β, IL-8 and tPA/PAI-2; IL-1β, IL-8, TIMP-1 and PAI-2; IL-1β, IL-8, TIMP-1 and tPA; tPA/PAI-2; PAI-2/tPA; IL-1β and PAI-2; in an ex vivo sample; and
b) determining the state of peri-implant disease by comparing the expression level obtained in step a) with one or more reference expression level(s).

2. The method according to claim 1, wherein said one or more regulated markers is selected from the group consisting of tPA and PAI-2, or any combination thereof, or any ratio thereof.

3. The method according to claim 1, wherein the expression level of the one or more regulated markers is the ratio between tPA and PAI-2.

4. The method according to claim 1, wherein said marker is tPA.

5. The method according to claim 1, wherein said one or more regulated markers is selected from the group consisting of IL-6, TIMP-1 and PAI-2, or any combination thereof, or any ratio thereof.

6. The method according to claim 1, wherein the ex vivo sample is a body fluid selected from an oral fluid, blood, serum, plasma, saliva or peri-implant crevicular fluid.

7-8. (canceled)

9. The method according to claim 16, wherein the ex vivo sample is a tissue selected from bone, connective tissue, mucosa, implant supporting tissue, bone adjacent to an implant, or bone adjacent to a tooth.

10. (canceled)

11. The method according to claim 1, wherein the one or more reference expression level(s) of step b) is the expression level(s) of said one or more regulated markers, or any combination thereof, or any ratio thereof from an ex vivo sample taken from the same source at a different point in time, and wherein an up-regulation or down-regulation of the expression levels of said one or more regulated markers, or any combination thereof, or any ratio thereof compared with the one or more reference expression level(s) is indicative of the state of peri-implant disease.

12. The method according to claim 1, wherein the one or more reference expression level(s) of step b) is the expression level of said one or more regulated markers, or any combination thereof, or any ratio thereof, from an ex vivo sample taken from a source showing a history of peri-implantitis, and wherein the up-regulation or down-regulation of the expression levels of said one or more regulated markers, or combination thereof, or ratio thereof compared with the one or more reference expression level(s) is indicative of the state of peri-implant disease.

13. The method according to claim 1, wherein quantifying the expression level is performed by a method for nucleic acid quantification selected from qPCR or Northern Blot.

14. (canceled)

15. The method according to claim 1, wherein quantifying the expression level is performed by a method for protein quantification selected from immunoassay, ELISA, radioimmunoassay, magnetic immunoassay, fluorescent immunoassay, immunoprecipitation, surface plasmon resonance, immunohistochemistry, Western Blot, or any combination thereof.

16. (canceled)

17. The method according to claim 1, wherein the ex vivo sample is obtained from a patient who has one or more implants, selected from a bone anchored implant, a dental implant, a hip implant, a knee implant, or a combination thereof.

18-19. (canceled)

20. The method according to claim 1, further comprising the step of saving the information regarding expression level of one or more regulated markers obtained in step (a) and/or saving the information regarding the state of peri-implant disease obtained in step (b) in a data carrier.

21. A kit for carrying out the method according to claim 1.

22. The kit according to claim 21, wherein the kit comprises a sample collecting device, selected from a sterile paper point, a syringe, or a biopsy device.

23. (canceled)

24. The kit according to claim 21, wherein the kit further comprises a preservation medium for preserving the sample.

25. The kit according to claim 21, wherein the kit further contains a box to send the sample to a central laboratory.

26. The kit according to claim 21, wherein the kit further contains the necessary elements to quantify the expression levels of one or more markers selected from the group consisting of IL-4, IL-6, IL-10, IL-12, IL18, TIMP-1, tPA and PAI-2 or any combination thereof, or any ratio thereof, or the necessary elements to quantify the expression levels of the following combinations of markers: tPA and tPA/PAI-2; PAI-2 and tPA/PAI-2; tPA, PAI-2 and tPA/PAI-2; tPA and IL-1β; tPA and TIMP-1; IL-1β and IL-8; IL-8 and PAI-2; IL-6, TIMP-1 and PAI-2; tPA and PAI-2; IL-1β, IL-8 and IL-6; IL-1β, IL-8, IL-6 and TIMP-1; IL-1β, IL-8, IL-6, TIMP-1 and tPA; IL-1β, IL-8, IL-6, TIMP-1 and PAI-2; IL-1β, IL-8, PAI-2 and tPA; IL-1β, IL-8, and PAI-2; IL-1β, IL-8 and tPA; IL-1β, IL-8 and tPA/PAI-2; IL-1β, IL-8, TIMP-1 and PAI-2; IL-1β, IL-8, TIMP-1 and tPA; tPA/PAI-2; PAI-2/tPA; IL-1β and PAI-2; or the necessary elements to quantify the expression levels of the following ratio of markers; tPA/PAI-2.

27. The kit according to claim 26, wherein said marker is tPA.

28. The kit according to claim 21, further comprising a data carrier.
